(19) United States
(12) Patent Application Publication
(10) Pub. No.: US 2026/0108458 A1
(43) Pub. Date: Apr. 23, 2026

(54) METHODS OF ADMINISTERING ENHANCED DELIVERY EPINEPHRINE AND PRODRUG COMPOSITIONS TO TREAT ALLERGY

(71) Applicant: Aquestive Therapeutics, Inc., Warren, NJ (US)

(72) Inventors: Stephen Paul Wargacki, Pittstown, NJ (US); Alexander Mark Schobel, Vero Beach, FL (US); Carl Neil Kraus, Raleigh, NC (US)

(73) Assignee: Aquestive Therapeutics, Inc., Warren, NJ (US)

(21) Appl. No.: 19/367,129

(22) Filed: Oct. 23, 2025

Related U.S. Application Data
(60) Provisional application No. 63/711,128, filed on Oct. 23, 2024.
(60) Provisional application No. 63/869,870, filed on Aug. 25, 2025.

(51) Int. Cl. A61K 9/00; A61K 31/05; A61K 31/11; A61K 31/137; A61K 31/192; A61K 31/198; A61K 31/222; A61K 31/231; A61K 31/36; A61K 31/765; A61K 36/61; A61K 47/10; A61K 47/14; A61K 47/26; A61K 47/32; A61K 47/38; A61P 37/08

(52) U.S. Cl. CPC A61K 9/006; A61K 31/05; A61K 31/11; A61K 31/137; A61K 31/192; A61K 31/198; A61K 31/222; A61K 31/231; A61K 31/36; A61K 31/765; A61K 36/61; A61K 47/10; A61K 47/14; A61K 47/26; A61K 47/32; A61K 47/38; A61P 37/08

(57) ABSTRACT

Methods of treating an allergic symptom by oral administration of epinephrine and its prodrugs are described as having pharmacokinetic parameters that are improved or comparable to those compositions administered by intramuscular injection.

### CLAIM OF PRIORITY

This application claims priority U.S. Provisional Patent Application No. 63/711,128, filed Oct. 23, 2024, and U.S. Provisional Patent Application No. 63/869,870, filed Aug. 25, 2025, each of which is incorporated by reference in its entirety.

### TECHNICAL FIELD

This invention relates to methods of administering pharmaceutical compositions.

### BACKGROUND

Active ingredients, such as drugs or pharmaceuticals, are delivered to patients in deliberate fashion. Delivery of drugs or pharmaceuticals using film transdermally or transmucosally can require that the drug or pharmaceutical permeate or otherwise cross a biological membrane in an effective and efficient manner.

### SUMMARY

In general, a method of treating an allergy symptom in a subject comprises applying a pharmaceutical film to a subject, the pharmaceutical film comprising a polymeric matrix to an oral cavity, a pharmaceutically active component including epinephrine or a pharmaceutically acceptable salt or ester thereof contained in the polymeric matrix and an adrenergic receptor interacter contained in the polymeric matrix; and substantially resolving the allergy symptom earlier than reaching a Cmax of the pharmaceutically active component.

In certain embodiments, the pharmaceutical film is applied proximate to a site of the allergy symptom.

In certain embodiments, the allergy symptom is substantially resolved prior to Tmax.

In certain embodiments, the onset of resolution of the allergy symptom occurs in 20 minutes or less. In certain embodiments, the onset of resolution of the allergy symptom occurs in 15 minutes or less. In certain embodiments, the onset of resolution of the allergy symptom occurs in 12 minutes or less.

In certain embodiments, the onset of resolution of the allergy symptom occurs in 10 minutes or less.

In certain embodiments, the onset of resolution of the allergy symptom occurs in 8 minutes or less.

In certain embodiments, the onset of resolution of the allergy symptom occurs in 5 minutes or less.

In certain embodiments, the onset of resolution of the allergy symptom occurs in 2 minutes or less.

In certain embodiments, the method further includes applying a repeat dose of the pharmaceutical film.

In certain embodiments, the allergy symptom is an oral allergy symptom.

In certain embodiments, the allergy symptom includes oral and body allergy symptoms.

In certain embodiments, the method further includes maintaining a consistent pharmacokinetic profile with a repeat dose.

In certain embodiments, the treatment elicits a desired pharmacodynamic response in systolic blood pressure, diastolic blood pressure and pulse with and without an allergen.

In certain embodiments, the adrenergic receptor interacter is a permeation enhancer that includes a phenylpropanoid, farnesol, a capric/caprylic triglyceride polyethylene glycol derivative or linoleic acid.

In certain embodiments, the adrenergic receptor interacter is eugenol or eugenol acetate, a cinnamic acid, cinnamic acid ester, cinnamic aldehyde, hydrocinnamic acid, chavicol, or safrole.

In certain embodiments, the adrenergic receptor interacter is a phytoextract.

In certain embodiments, the phytoextract further includes an essential oil extract of a clove plant, an essential oil extract of a leaf of a clove plant, an essential oil extract of a flower bud of a clove plant, or an essential oil extract of a stem of a clove plant.

In certain embodiments, the phytoextract is a synthetic or biosynthetic compound.

In certain embodiments, a Cmax of the pharmaceutically active component is between 250-500 pg/mL.

In certain embodiments, the Tmax of the pharmaceutically active component is 15 minutes. In certain embodiments, the Tmax of the pharmaceutically active component is 12 minutes. In certain embodiments, the Tmax of the pharmaceutically active component is 10 minutes. In certain embodiments, the Tmax of the pharmaceutically active component is 8 minutes. In certain embodiments, the Tmax of the pharmaceutically active component is 5 minutes.

In certain embodiments, the Cmax is greater than 34 pg/mL. In certain embodiments, the Cmax is greater than 70 pg/mL. In certain embodiments, the Cmax is greater than 150 pg/mL. In certain embodiments, the Cmax is greater than 300 pg/mL. In certain embodiments, the Cmax is greater than 450 pg/mL. In certain embodiments, the Cmax is less than 2850 pg/mL. In certain embodiments, the Cmax is in the range of 34-5000 pg/mL

In certain embodiments the pharmacokinetic parameter is Tmax. In certain embodiments, the Tmax is greater than 8 minutes. In certain embodiments, the Tmax is about 12 minutes. In certain embodiments, the Tmax is about 12-15 minutes. In certain embodiments, the Tmax is greater than 15 minutes. In certain embodiments, the Tmax is greater than 25 minutes. In certain embodiments, the Tmax is greater than 40 minutes. In certain embodiments, the Tmax is greater than 50 minutes. In certain embodiments, the Tmax is 8-50 minutes.

In certain embodiments, the composition further includes a mixture of adrenergic receptor interacters.

In certain embodiments, the adrenergic receptor interacter includes an aromatic compound. In certain embodiments, the adrenergic receptor interacter includes a phenylpropanoid. In certain embodiments, the adrenergic receptor interacter includes farnesol or Labrasol. In certain embodiments, the adrenergic receptor interacter includes linoleic acid.

In certain embodiments, the adrenergic receptor interacter includes a phenylpropanoid that is eugenol or eugenol acetate. In certain embodiments, the phenylpropanoid is a cinnamic acid, cinnamic acid ester, cinnamic aldehyde or hydrocinnamic acid. In certain embodiments, the phenylpropanoid is chavicol. In certain embodiments, the phenylpropanoid is safrole.

In certain embodiments, the adrenergic receptor interacter is a phytoextract. In certain embodiments, the phytoextract is synthetic or biosynthetic. In certain embodiments, the phytoextract further includes an essential oil extract of a clove plant.

In general, a method of administering a pharmaceutical composition, includes providing an oral film including: a polymeric matrix; a pharmaceutically active component including epinephrine or its prodrug in the polymeric matrix; and an adrenergic receptor interacter; positioning the film in an oral mucosa for a residence time; and allowing the film to deliver the pharmaceutically active component.

In certain embodiments, the residence time is no less than 4 minutes. In certain embodiments, the residence time is no less than 2 minutes. In certain embodiments, the residence time is no less than 0 minutes.

In certain embodiments, the residence time is no more than 2 minutes. In certain embodiments, the residence time is no more than 4 minutes. In certain embodiments, the residence time is no more than 6 minutes. In certain embodiments, the residence time is no more than 8 minutes.

In certain embodiments, the film is placed on the sublingual mucosa, pressed down on the mucosa, held down on the mucosa, applied to the underside of the tongue.

After initial placement, the film may move under the tongue and throughout the sublingual space contacting teeth and or gums without impacting the performance of the film. During disintegration, saliva may be held for 5 minutes, 4 minutes, 3 minutes, 2 minutes, 1 minute, or not at all. The film can be administered in the presence of solid or liquid residue. The film can be administered in the presence of edema such as a reaction to the presence of allergens.

In certain embodiments, the AUC for 0-10 minutes is equal to or greater than an intramuscular injection of a composition having a same pharmaceutically active component.

In certain embodiments, the AUC for 0-10 minutes is greater than 10 hr*pg/mL for a 4-minutes residence time. In certain embodiments, the AUC for 0-10 minutes is about 9 hr*pg/mL for a 2-minute residence time. In certain embodiments, the AUC for 0-10 minutes is about 9 hr*pg/mL for a 0-minute residence time.

In certain embodiments, the AUC for 0-20 minutes is equal to or greater than an intramuscular injection of a composition having a same pharmaceutically active component. In certain embodiments, the AUC for 0-20 minutes is about 50 hr*pg/mL for a 4-minute residence time.

In certain embodiments, the AUC for 0-20 minutes is about 45 hr*pg/mL for a 2-minute residence time. In certain embodiments, the AUC for 0-20 minutes is about 30 hr*pg/mL for a 0-minute residence time.

In certain embodiments, the AUC for 0-30 minutes is equal to or greater than an intramuscular injection of a composition having a same pharmaceutically active component.

In certain embodiments, the AUC for 0-30 minutes is about 80 hr*pg/mL for a 4-minute residence time. In certain embodiments, the AUC for 0-30 minutes is about 50 hr*pg/mL for a 2-minute residence time. In certain embodiments, the AUC for 0-30 minutes is about 50 hr*pg/mL for a 0-minute residence time.

In certain embodiments, therapeutic levels of active can be achieved in less than 15 minutes, less than 10 minutes or less than 5 minutes.

In certain embodiments, the composition is a film further comprising a polymeric matrix, the pharmaceutically active component being contained in the polymeric matrix.

In certain embodiments, the adrenergic receptor interacter includes a phenylpropanoid.

In certain embodiments, the adrenergic receptor interacter includes farnesol or Labrasol. In certain embodiments, the adrenergic receptor interacter includes linoleic acid. In certain embodiments, the adrenergic receptor interacter includes an aromatic compound, such as a substituted aromatic alcohol, for example, 8-hydroxy-p-cymene. In certain embodiments, the phenylpropanoid is eugenol or eugenol acetate. In certain embodiments, the phenylpropanoid is a cinnamic acid, cinnamic acid ester, cinnamic aldehyde or hydrocinnamic acid. In certain embodiments, the phenylpropanoid is chavicol. In certain embodiments, the phenylpropanoid is safrole. In certain embodiments, the adrenergic receptor interacter is a phytoextract. In certain embodiments, the phytoextract is synthetic or biosynthetic. In certain embodiments, the phytoextract further includes an essential oil extract of a clove plant.

In certain embodiments, the phytoextract further includes 40-95% eugenol. In certain embodiments, the adrenergic receptor interacter includes a terpenoid, terpene or a sesquiterpene. In certain embodiments, the polymer matrix includes a polymer. In certain embodiments,

the polymer is a water soluble polymer. In certain embodiments, the polymer includes a polyethylene oxide. In certain embodiments, the polymer includes a cellulosic polymer is selected from the group of: hydroxypropylmethyl cellulose, hydroxyethyl cellulose, hydroxyethylmethyl cellulose, hydroxypropyl cellulose, methylcellulose and carboxymethyl cellulose. In certain embodiments, the polymeric matrix comprises a cellulosic polymer, polyethylene oxide and polyvinyl pyrrolidone, polyethylene oxide and a polysaccharide, polyethylene oxide, hydroxypropyl methylcellulose and a polysaccharide, or polyethylene oxide, hydroxypropyl methylcellulose, polysaccharide and polyvinylpyrrolidone.

In certain embodiments, the polymeric matrix comprises at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

In certain embodiments, the method further comprises a stabilizer. In certain embodiments, the polymeric matrix comprises a dendritic polymer or a hyperbranched polymer.

In certain embodiments, the prodrug is dipifevrin.

In certain embodiments, swallowing is permitted after the residence time. In certain embodiments, the pharmaceutically active component includes a prodrug of epinephrine.

In certain embodiments, the prodrug of epinephrine has a half-life of less than one minute.

In general, a method of administering epinephrine to a subject includes self-administering a composition including a prodrug of epinephrine to the oral cavity of the subject to deliver epinephrine through a digestive tract of the subject. In certain embodiments, the composition is an oral film. In certain embodiments, the composition further includes an adrenergic receptor interacter. In certain embodiments, the composition further includes a mixture of two adrenergic receptor interacters. In certain embodiments, the composition further includes a mixture of three adrenergic receptor interacters.

In certain embodiments, the oral film can include an absorbant component, an adsorbant component, or a desiccant. In certain embodiments, a suitable absorbant component, adsorbant component, or desiccant can include a silica, fumed silica or a mesoporous silica. In certain embodiments, the absorbant component, adsorbant component, or desiccant can be an amorphous silicon dioxide. In certain embodiments, the absorbant component, adsorbant component, or desiccant can include silicon dioxide variants such as a Cab-o-Sil or a Syloid product. In certain embodiments, the absorbant component, adsorbant component, or desiccant can be 1-15% by weight of the pharmaceutical composition. For example, the desiccant can be 2%, 3%, 4%, 5%, 6%, 7%, 8%, 9%, 10%, 11%, 12%, 13%, 14%, or 15% w/w of the pharmaceutical composition. In certain embodiments, the silicon dioxide can have an average surface area of about 25-600 m²/g, for example 25 m²/g, 50 m²/g, 100 m²/g, 200 m²/g, 300 m²/g, 400 m²/g, 500 m²/g, or 600 m²/g. In certain embodiments, the silicon dioxide can have an average particle size of about 1-10 microns, for example 1 micron, 2 microns, 4 microns, 5 microns, 8 microns, about 10 microns or greater. In certain embodiments, one or more oil components of the composition can be adsorbed on the desiccant. In certain embodiments, the absorbant component, the adsorbant component, or desiccant can adsorb up to about 0.5 mL, 1.0 mL, 1.5 mL, or 2.0 mL of liquid per gram of desiccant.

In certain embodiments, the composition is formulated to be absorbed through the stomach, intestine or gastrointestinal tract. In certain embodiments, the composition is administered up to 2 minutes after consuming food. In certain embodiments, the composition is administered up to 4 minutes after consuming food. In certain embodiments, the composition is administered up to 6 minutes after consuming food. In certain embodiments, the composition is administered up to 8 minutes after consuming food. In certain embodiments, the composition is administered up to 10 minutes after consuming food. In certain embodiments, the composition is administered up to 15 minutes after consuming food. In certain embodiments, the composition is administered up to 20 minutes after consuming food. In certain embodiments, the composition is administered up to 30 minutes after consuming food. In certain embodiments, the composition is administered up to 60 minutes after consuming food. In certain embodiments, the composition is administered without food or drink. In certain embodiments, the composition is swallowed after an oral cavity residence or retention time. In certain embodiments, the composition is swallowed without a residence or retention time.

In certain embodiments, the residence or retention time is no less than 15 minutes. In certain embodiments, the residence or retention time is no less than 10 minutes. In certain embodiments, the residence or retention time is no less than 8 minutes. In certain embodiments, the residence or retention time is no less than 6 minutes. In certain embodiments, the residence or retention time is no less than 4 minutes. In certain embodiments, the residence or retention time is no less than 2 minutes. In certain embodiments, the residence or retention time is less than 2 minutes. In certain embodiments, the AUC for 0-10 minutes is greater than 10 hr*pg/mL for a 4-minutes residence time.

In certain embodiments, the AUC for 0-10 minutes is about 15 hr*pg/mL after food intake. In certain embodiments, the AUC for 0-10 minutes is about 2 hr*pg/mL with no residence time. In certain embodiments, the AUC for 0-20 minutes is about 50 hr*pg/mL for a 4-minute residence time. In certain embodiments, the AUC for 0-20 minutes is about 49 hr*pg/mL after food intake. In certain embodiments, the AUC for 0-20 minutes is about 28 hr*pg/mL with no residence time. In certain embodiments, the AUC for 0-30 minutes is about 79 hr*pg/mL for a 4-minute residence time. In certain embodiments, the AUC for 0-30 minutes is about 65 hr*pg/mL after food intake. In certain embodiments, the AUC for 0-30 minutes is about 73 hr*pg/mL with no residence time.

Other aspects, embodiments, and features will be apparent from the following description, the drawings, and the claims.

### BRIEF DESCRIPTION OF THE FIGURES

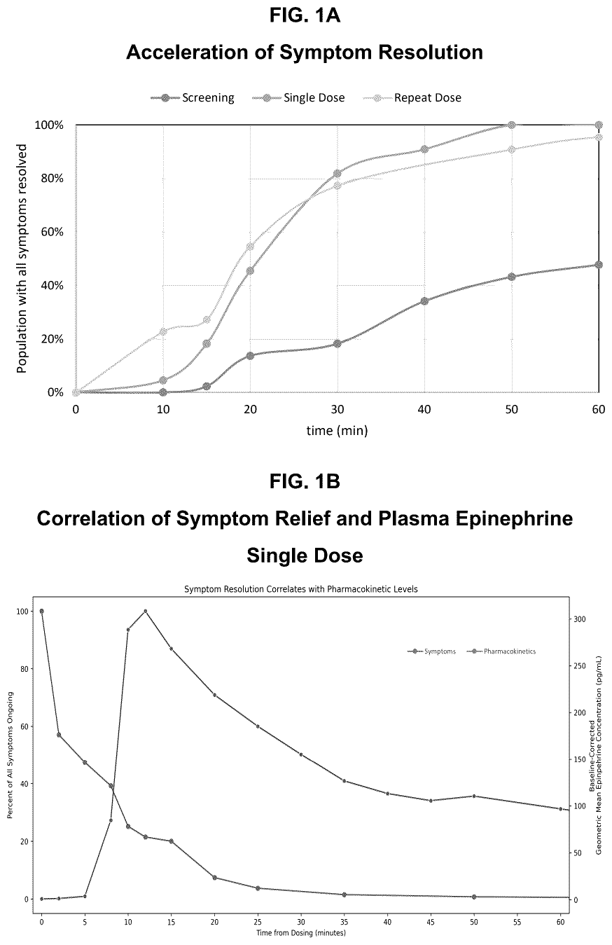

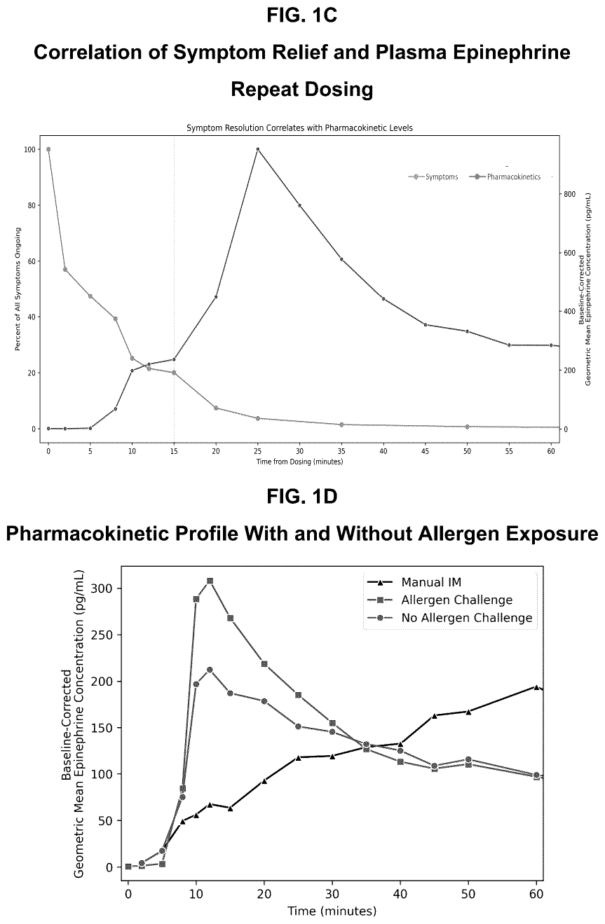

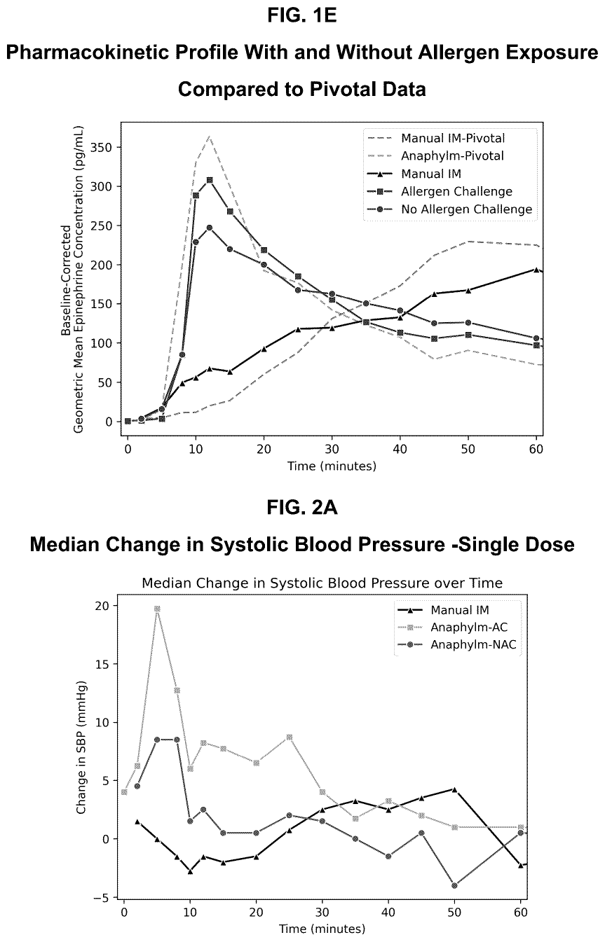

Referring to FIG. 1A, this shows the acceleration of symptom resolution after the method of treatment with the pharmaceutical composition.

Referring to FIG. 1B, this shows the correlation of symptom relief and plasma epinephrine levels (pg/ml) after a single treatment.

Referring to FIG. 1C, this shows the correlation of symptom relief and plasma epinephrine levels (pg/ml) after a repeat treatment.

Referring to FIG. 1D, this shows a pharmacokinetic profile of epinephrine levels over time with and without allergen exposure.

Referring to FIG. 1E, this shows a pharmacokinetic profile of epinephrine levels over time with and without allergen exposure compared to pivotal data.

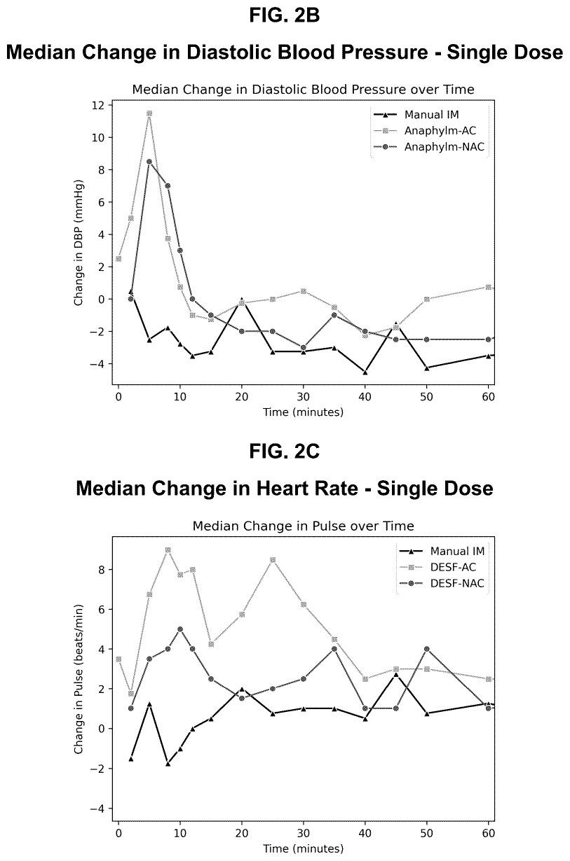

Referring to FIG. 2A, this shows the median change in systolic blood pressure over time after a single dose.

Referring to FIG. 2B, this shows the median change in diastolic blood pressure over time after a single dose.

Referring to FIG. 2C, this shows the median change in heart rate time after a single dose

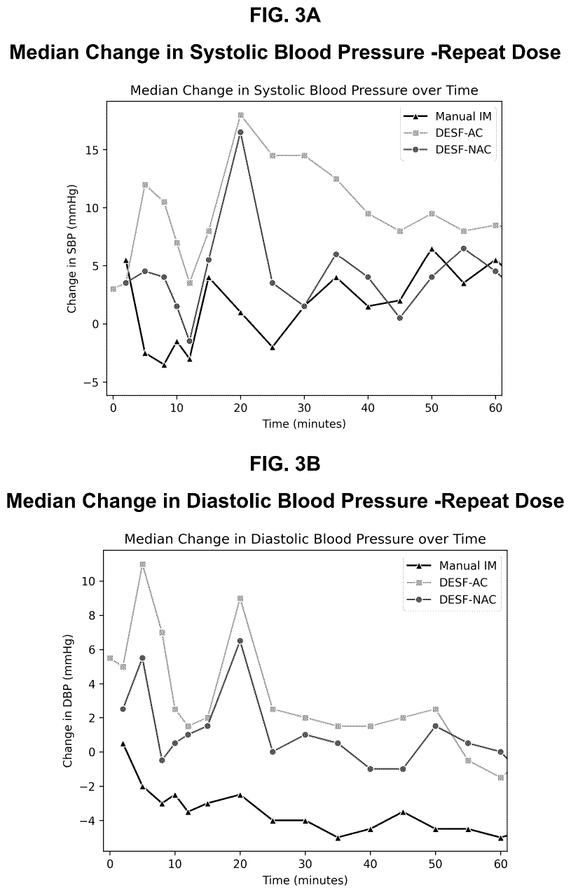

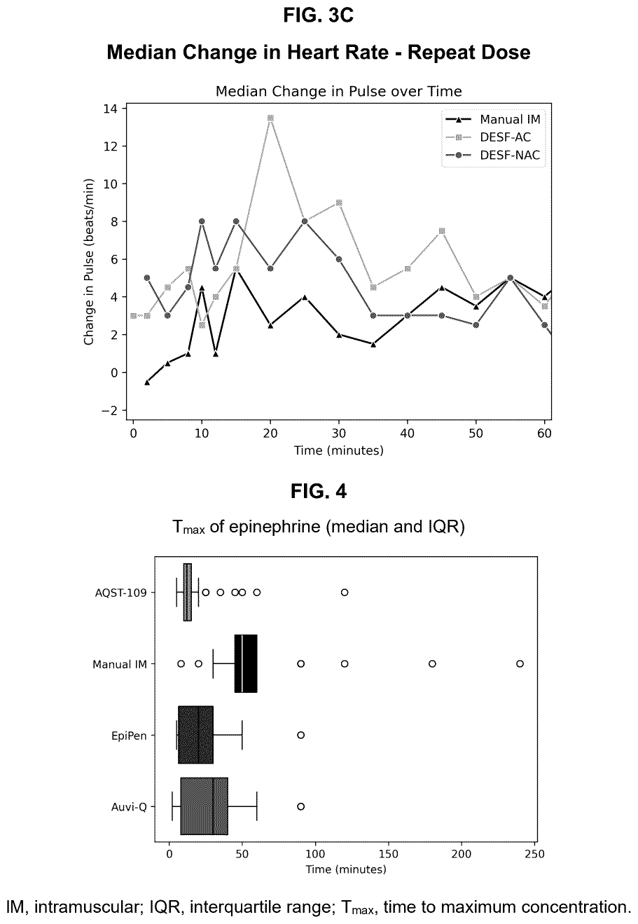

Referring to FIG. 3A, this shows the median change in systolic blood pressure over time after a repeat dose.

Referring to FIG. 3B, this shows the median change in diastolic blood pressure over time after a repeat dose.

Referring to FIG. 3C, this shows the median change in diastolic blood pressure over time after a repeat dose.

Referring to FIG. 4, this shows measured Tmax of a pharmaceutical film compared to EpiPen®, Auvi-Q® and manual injection.

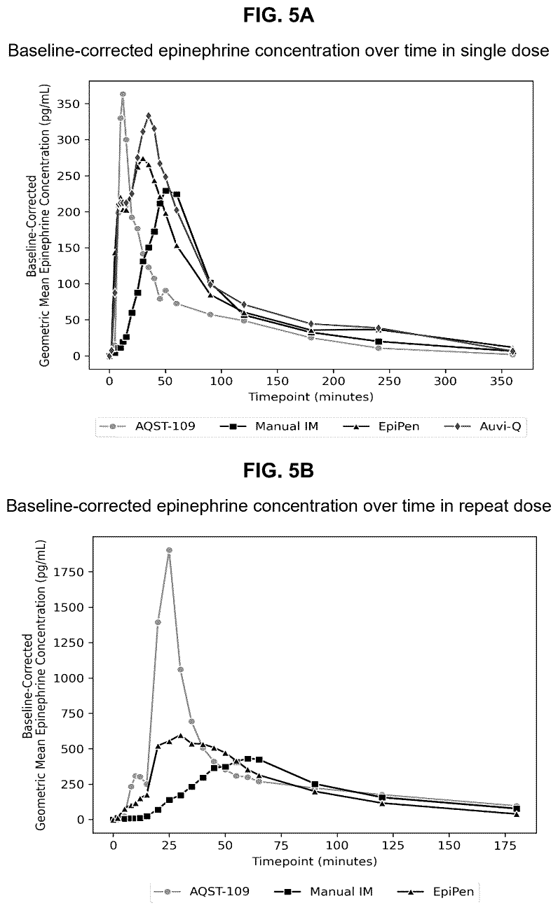

Referring to FIG. 5A, this shows baseline-corrected epinephrine concentration over time in a single dose.

Referring to FIG. 5B, this shows baseline-corrected epinephrine concentration over time in a single dose.

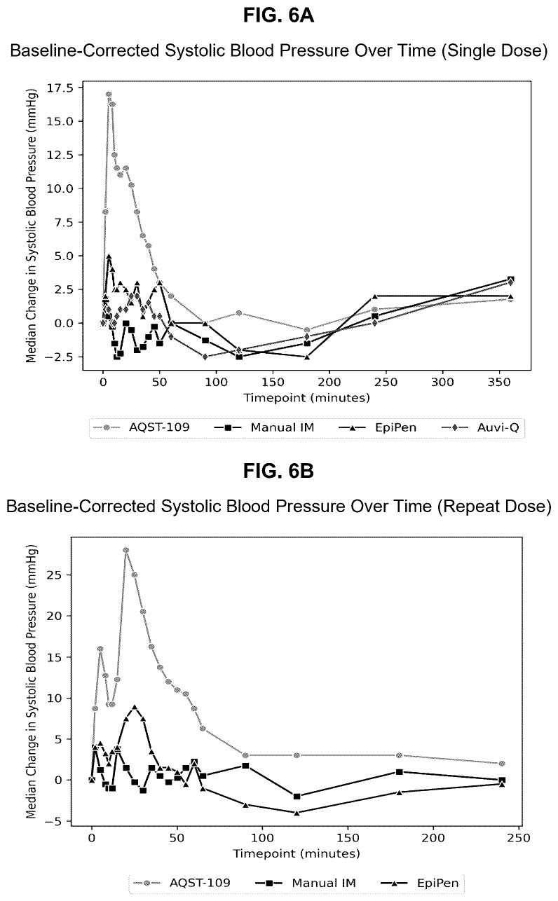

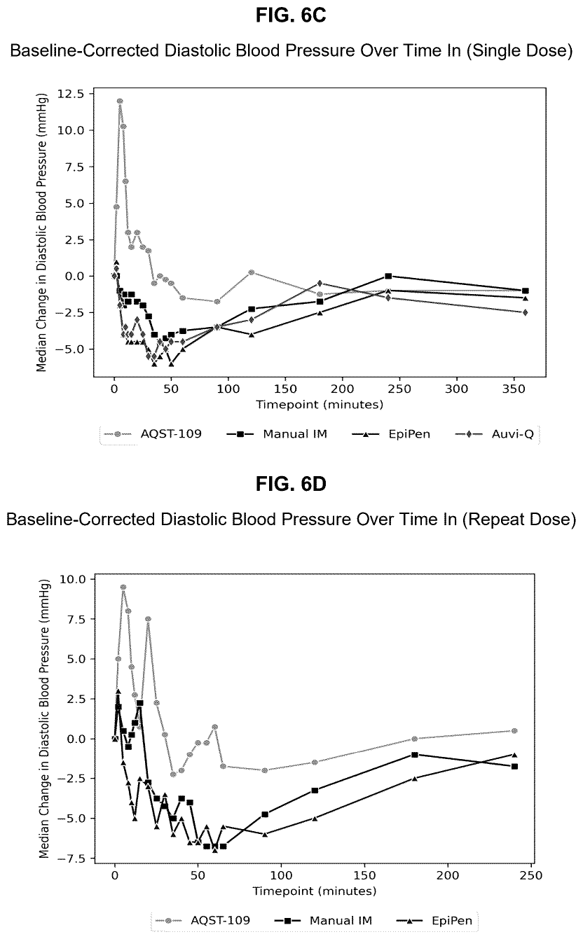

Referring to FIG. 6A, this shows baseline-corrected systolic blood pressure over time in a single dose.

Referring to FIG. 6B, this shows baseline-corrected systolic blood pressure over time in a repeat dose.

Referring to FIG. 6C, this shows baseline-corrected diastolic blood pressure over time in a single dose.

Referring to FIG. 6D, this shows baseline-corrected diastolic blood pressure over time in a repeat dose.

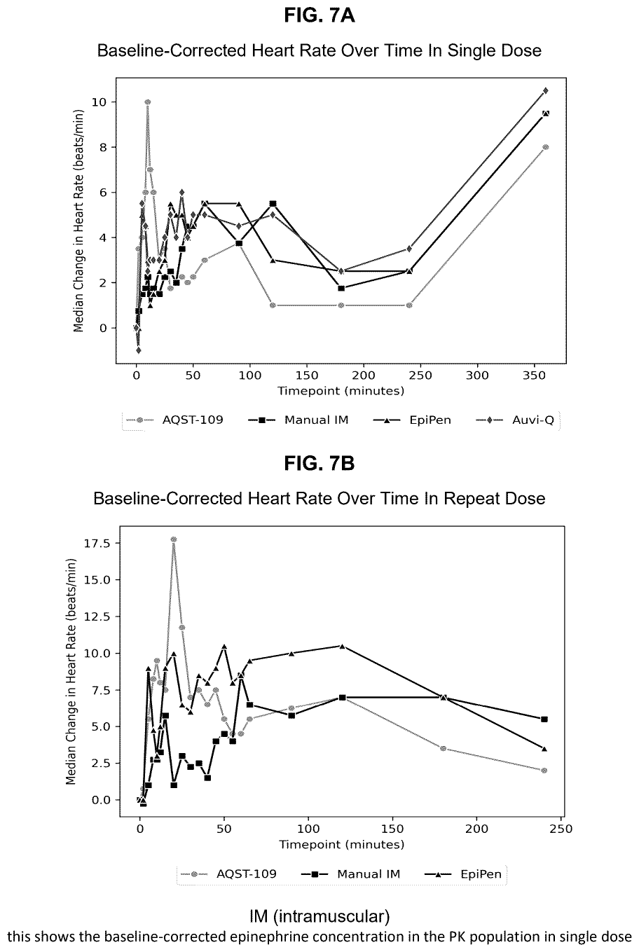

Referring to FIG. 7A, this shows baseline-corrected heart rate over time in a single dose.

Referring to FIG. 7B, this shows baseline-corrected heart rate over time in a repeat dose.

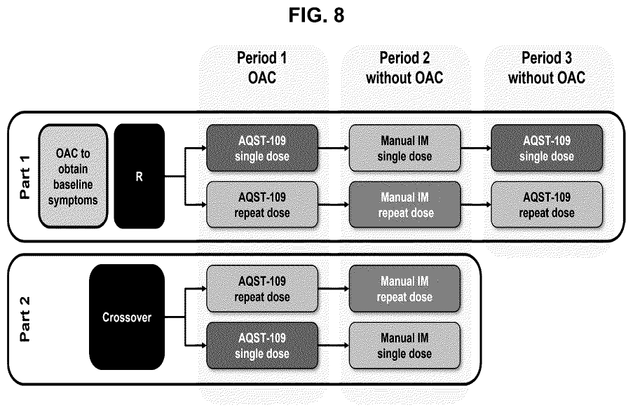

Referring to FIG. 8, this shows the schematic of a Phase 2 study with the claimed pharmaceutical composition.

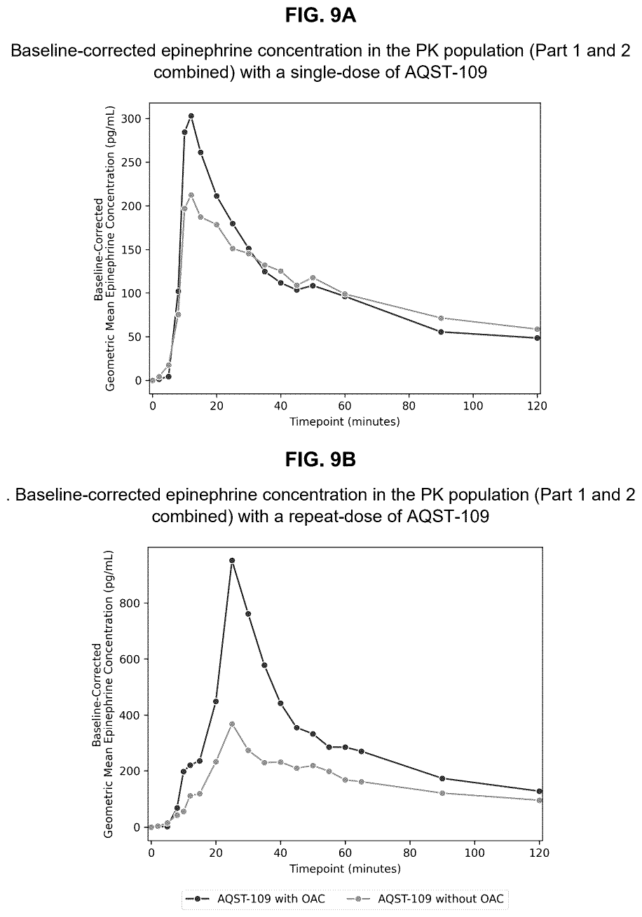

Referring to FIG. 9A, this shows the baseline-corrected epinephrine concentration in the PK population in a single dose.

Referring to FIG. 9B, this shows the baseline-corrected epinephrine concentration in the PK population in a repeat dose.

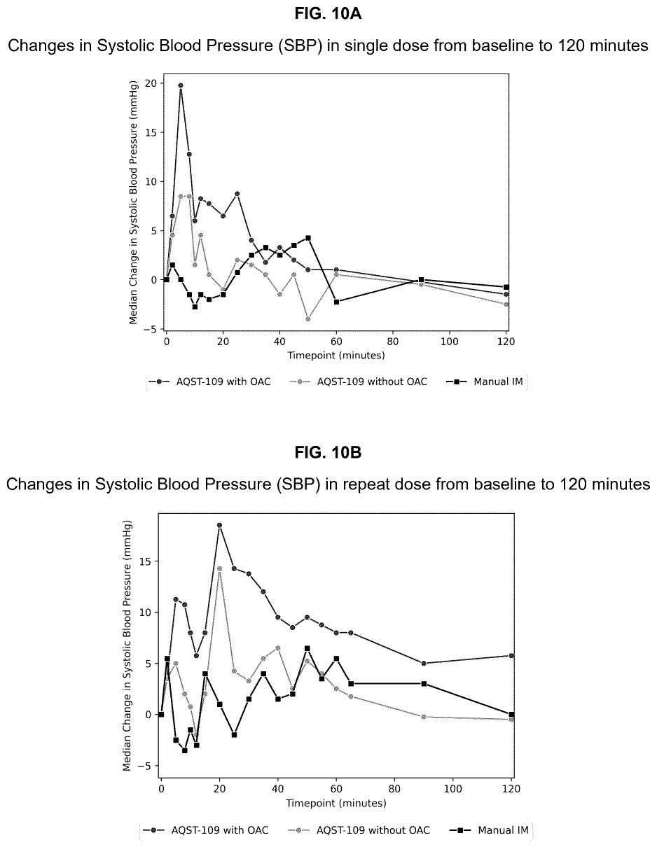

Referring to FIG. 10A, this shows changes in systolic blood pressure in a single dose from baseline to 120 minutes.

Referring to FIG. 10B, this shows changes in systolic blood pressure in a repeat dose from baseline to 120 minutes.

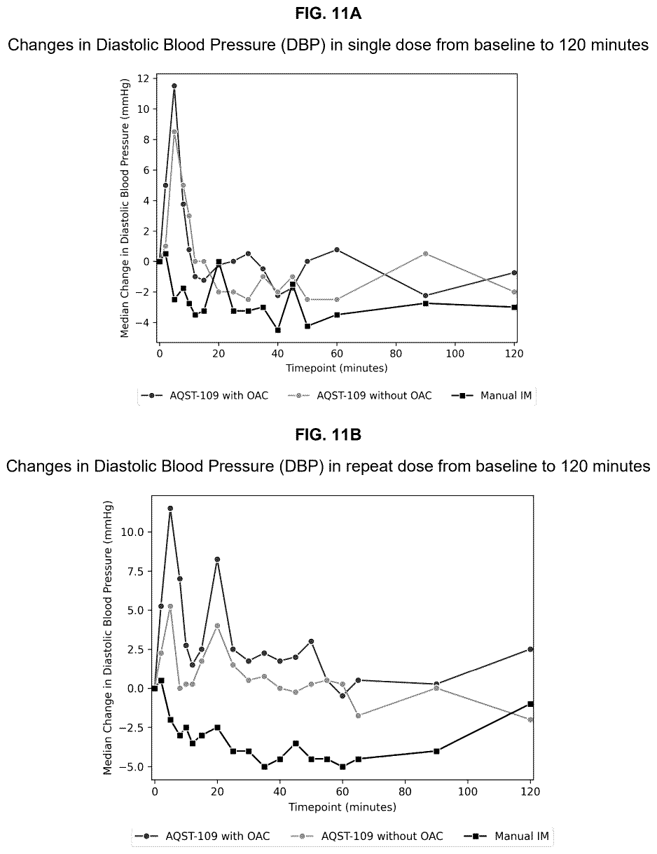

Referring to FIG. 11A, this shows changes in diastolic blood pressure in a single dose from baseline to 120 minutes.

Referring to FIG. 11B, this shows changes in diastolic blood pressure in a repeat dose from baseline to 120 minutes.

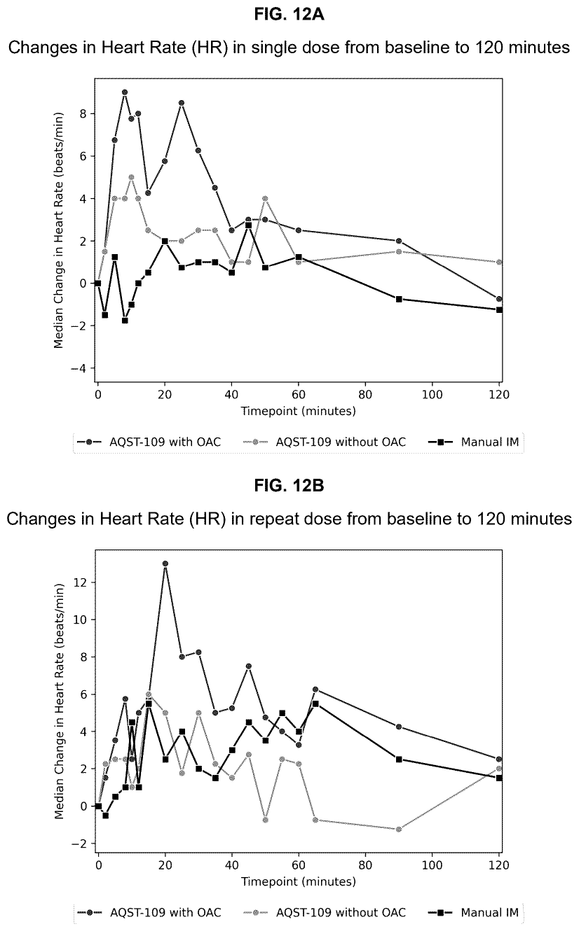

Referring to FIG. 12A, this shows changes in heart rate in a single dose from baseline to 120 minutes.

Referring to FIG. 12B, this shows changes in heart rate in a repeat dose from baseline to

120 minutes.

### DETAILED DESCRIPTION

Pharmaceutical film including a prodrug of epinephrine can be administered to a subject experiencing an allergic symptom can provide relief in a short timeframe after administration. For example, administration can lead to substantial resolution of a symptom by reducing the severity of the symptom or alleviating the symptom in a subject. Subjects categorized as having moderate or severe symptoms after exposure to an allergen received an administration of epinephrine sublingual film which resulted in rapid symptom relief in as little as two minutes. The median time to full symptom resolution after administration was about 12 minutes (includes single and repeat dose administrations).

In certain embodiments, the method includes substantially resolving the allergy symptom earlier than reaching a Cmax of the pharmaceutically active component. Moreover, the pharmaceutical film can be applied proximate to a site of the allergy symptom, which can improve the onset of resolution of symptoms.

In certain embodiments, the allergy symptom can be substantially resolved prior to Tmax. Noticeable onset of resolution of the allergy symptom can occur in 20 minutes or less, 15 minutes or less, 12 minutes or less, 10 minutes or less, 8 minutes or less, 5 minutes or less, or 2 minutes or less.

In certain circumstances, applying a repeat dose of the pharmaceutical film can aid in reducing or resolving the symptom.

In certain circumstances, the allergy symptom can be an oral allergy symptom, body allergy symptom, or systemic allergy symptom.

Mucosal surfaces, such as the oral mucosa, are a convenient route for delivering drugs to the body due to the fact that they are highly vascularized and permeable, providing increased bioavailability and rapid onset of action because it does not pass through the digestive system and thereby may avoid first pass metabolism. In particular, the buccal and sublingual tissues offer advantageous sites for drug delivery because they are highly permeable regions of the oral mucosa, allowing drugs diffusing from the oral mucosa to have direct access to systemic circulation. This also offers increased convenience and therefore increased compliance in patients. For certain drugs, or pharmaceutically active components, a permeation enhancer can help to overcome the mucosal barrier and improve permeability. Permeation enhancers reversibly modulate the penetrability of the barrier layer in favor of drug absorption. Permeation enhancers facilitate transport of molecules through the epithelium. Absorption profiles and their rates can be controlled and modulated by a variety of parameters, such as but not limited to film size, drug loading, enhancer type/loading, polymer matrix release rate and mucosal residence time.

A pharmaceutical composition can be designed to deliver a pharmaceutically active component in a deliberate and tailored way. However, solubility and permeability of the pharmaceutically active component in vivo, in particular, in the mouth of a subject, can vary tremendously. A particular class of permeation enhancer can improve the uptake and bioavailability of the pharmaceutically active component in vivo. In particular, when delivered to the mouth via a film, the permeation enhancer can improve the permeability of the pharmaceutically active component through the mucosa and into the blood stream of the subject. The permeation enhancer can improve absorption rate and amount of the pharmaceutically active component by more than 5%, more than 10%, more than 20%, more than 30%, more than 40%, more than 50%, more than 60%, more than 70%, more than 80%, more than 90%, more than 100%, more than 150%, about 200% or more, or less than 200%, less than 150%, less than 100%, less than 90%, less than 80%, less than 70%, less than 60%, less than 50%, less than 40%, less than 30%, less than 20%, less than 10%, or less than 5%, or a combination of these ranges, depending on the other components in the composition.

In certain embodiments, a pharmaceutical composition has a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced transmucosal delivery, wherein the formulation of the compound with the transmucosal delivery-enhancing agents provides for increased bioavailability of the compound in blood plasma of a subject. Penetration enhancers have been described in J. Nicolazzo, et al., J. of Controlled Disease, 105 (2005) 1-15, which is incorporated by reference herein.

There are many reasons why the oral mucosa might be an attractive site for the delivery of therapeutic agents into the systemic circulation. Due to the direct drainage of blood from the buccal epithelium into the internal jugular vein first-pass metabolism in the liver and intestine may be avoided. First-pass effect can be a major reason for the poor bioavailability of some compounds when administered orally. Additionally, the mucosa lining the oral cavity is easily accessible, which ensures that a dosage form can be applied to the required site and can be removed easily in the case of an emergency. However, like the skin, the buccal mucosa acts as a barrier to the absorption of xenobiotics, which can hinder the permeation of compounds across this tissue. Consequently, the identification of safe and effective penetration enhancers has become a major goal in the quest to improve oral mucosal drug delivery.

A residence time refers to a time period in which a pharmaceutical composition, such as a pharmaceutical film, may be held in a given position on an oral mucosa before swallowing or without swallowing. The residence time can allow a fast dissolving dosage form to be a versatile platform for drug delivery, and can result in systemic and local action via oral, buccal, and/or sublingual, routes.

Chemical penetration enhancers are substances that control the permeation rate of a coadministered drug through a biological membrane. While extensive research has focused on obtaining an improved understanding of how penetration enhancers might alter intestinal and transdermal permeability, far less is known about the mechanisms involved in buccal and sublingual penetration enhancement.

The buccal mucosa delineates the inside lining of the cheek as well as the area between the gums and upper and lower lips and it has an average surface area of 100 cm². The surface of the buccal mucosa consists of a stratified squamous epithelium which is separated from the underlying connective tissue (lamina propria and submucosa) by an undulating basement membrane (a continuous layer of extracellular material approximately 1-2 μm in thickness). This stratified squamous epithelium consists of differentiating layers of cells which change in size, shape, and content as they travel from the basal region to the superficial region, where the cells are shed. There are approximately 40-50 cell layers, resulting in a buccal mucosa which is 500-600 μm thick.

Structurally the sublingual mucosa is comparable to the buccal mucosa but the thickness of this epithelium is 100-200 pm. This membrane is also non-keratinised and being relatively thinner has been demonstrated to be more permeable than buccal mucosa. Blood flow to the sublingual mucosal is slower compared with the buccal mucosa and is of the order of 1.0 ml/min-1/cm-2.

The permeability of the buccal mucosa is greater than that of the skin, but less than that of the intestine. The differences in permeability are the result of structural differences between each of the tissues. The absence of organized lipid lamellae in the intercellular spaces of the buccal mucosa results in greater permeability of exogenous compounds, compared to keratinized epithelia of the skin; while the increased thickness and lack of tight junctions results in the buccal mucosa being less permeable than intestinal tissue.

The primary barrier properties of the buccal mucosa have been attributed to the upper one-third to one-quarter of the buccal epithelium. Researchers have learned that beyond the surface epithelium, the permeability barrier of nonkeratinized oral mucosa could also be attributed to contents extruded from the membrane-coating granules into the epithelial intercellular spaces.

The intercellular lipids of the nonkeratinized regions of the oral cavity are of a more polar nature than the lipids of the epidermis, palate, and gingiva, and this difference in the chemical nature of the lipids may contribute to the differences in permeability observed between these tissues. Consequently, it appears that it is not only the greater degree of intercellular lipid packing in the stratum corneum of keratinized epithelia that creates a more effective barrier, but also the chemical nature of the lipids present within that barrier.

The existence of hydrophilic and lipophilic regions in the oral mucosa has led researchers to postulate the existence of two routes of drug transport through the buccal mucosa paracellular (between the cells) and transcellular (across the cells).

Since drug delivery through the buccal mucosa is limited by the barrier nature of the epithelium and the area available for absorption, various enhancement strategies are required in order to deliver therapeutically relevant amounts of drug to the systemic circulation. Various methods, including the use of chemical penetration enhancers, prodrugs, and physical methods may be employed to overcome the barrier properties of the buccal mucosa.

A chemical penetration enhancer, or absorption promoter, is a substance added to a pharmaceutical formulation in order to increase the membrane permeation or absorption rate of the coadministered drug, without damaging the membrane and/or causing toxicity. There have been many studies investigating the effect of chemical penetration enhancers on the delivery of compounds across the skin, nasal mucosa, and intestine. In recent years, more attention has been given to the effect of these agents on the permeability of the buccal mucosa. Since permeability across the buccal mucosa is considered to be a passive diffusion process the steady state flux (Jss) should increase with increasing donor chamber concentration (CD) according to Fick's first law of diffusion.

Surfactants and bile salts have been shown to enhance the permeability of various compounds across the buccal mucosa, both in vitro and in vivo. The data obtained from these studies strongly suggest that the enhancement in permeability is due to an effect of the surfactants on the mucosal intercellular lipids.

Fatty acids have been shown to enhance the permeation of a number of drugs through the skin, and this has been shown by differential scanning calorimetry and Fourier transform infrared spectroscopy to be related to an increase in the fluidity of intercellular lipids.

Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azone® on the permeability of compounds through oral mucosa. Further, chitosan, a biocompatible and biodegradable polymer, has been shown to enhance drug delivery through various tissues, including the intestine and nasal mucosa.

While intramuscular injection can adequately deliver epinephrine in patients experiencing anaphylaxis, a different route of administration could address significant unmet needs that have led to marked under-utilization of this life-saving treatment. Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery-Current status and future prospects, Int'l. Journal of Pharmaceutics, 47 (2014) 498-506, which is incorporated by reference herein. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium, as well as the emergence of new analytical techniques to study the oral cavity, and the progressing development of in silico models predictive of buccal and sublingual permeation, prospects are encouraging.

In order to deliver broader classes of drugs across the buccal mucosa, reversible methods of reducing the barrier potential of this tissue should be employed. This requisite has fostered the study of penetration enhancers that will safely alter the permeability restrictions of the buccal mucosa. It has been shown that buccal penetration can be improved by using various classes of transmucosal and transdermal penetration enhancers such as bile salts, surfactants, fatty acids and their derivatives, chelators, cyclodextrins and chitosan. Among these chemicals used for the drug permeation enhancement, bile salts are the most common.

In vitro studies on enhancing effect of bile salts on the buccal permeation of compounds is discussed in Sevda Senel, Drug permeation enhancement via buccal route: possibilities and limitations, Journal of Controlled Release 72 (2001) 133-144, which is incorporated by reference herein. That article also discusses recent studies on the effects of buccal epithelial permeability of dihydroxy bile salts, sodium glycodeoxycholate (SGDC) and sodium taurodeoxycholate (TDC) and tri-hydroxy bile salts, sodium glycocholate (GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, 8-hydroxy-p-cymene, or safrole, or a combination thereof. The phytoextract can be an essential oil extract of a clove plant, for example, from the leaf, stem or flower bud of a clove plant. The clove plant can be Syzygium aromaticum. The phytoextract can include 20-95% eugenol, including 40-95% eugenol, including 60-95% eugenol, and for example, 80-95% eugenol. The extract can also include 5% to 15% eugenol acetate. The extract can also include caryophyllene. The extract can also include up to 2.1% α-humulen. Other volatile compounds included in lower concentrations in clove essential oil can be β-pinene, limonene, farnesol, benzaldehyde, 2-heptanone and ethyl hexanoate. Other permeation enhancers may be added to the composition to improve absorption of the drug. Suitable permeation enhancers include natural or synthetic bile salts such as sodium fusidate; glycocholate or deoxycholate and their salts; fatty acids and derivatives such as sodium laurate, oleic acid, oleyl alcohol, monoolein, and palmitoylcarnitine; chelators such as disodium EDTA, sodium citrate and sodium laurylsulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol. The permeation enhancer can include phytoextract derivatives and/or monolignols. The permeation enhancer can also be a fungal extract.

Some natural products of plant origin have been known to have a vasodilatory effect. There are several mechanisms or modes by which plant-based products can evoke vasodilation. For review, see McNeill J. R. and Jurgens, T. M., Can. J. Physiol. Pharmacol. 84:803-821 (2006), which is incorporated by reference herein. Specifically, vasorelaxant effects of eugenol have been reported in a number of animal studies. See, e.g., Lahlou, S., et al., J. Cardiovasc. Pharmacol. 43:250-57 (2004), Damiani, C. E. N., et al., Vascular Pharmacol. 40:59-66 (2003), Nishijima, H., et al., Japanese J. Pharmacol. 79:327-334 (1998), and Hume W. R., J. Dent Res. 62 (9): 1013-15 (1983), each of which is incorporated by reference herein. Calcium channel blockade was suggested to be responsible for vascular relaxation induced by a plant essential oil, or its main constituent, eugenol. See, Interaminense L. R. L. et al., Fundamental & Clin. Pharmacol. 21:497-506 (2007), which is incorporated by reference herein.

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference herein. There are two families of essential fatty acids that are metabolized in the human body: @-3 and @-6 polyunsaturated fatty acids (PUFAs). If the first double bond is found between the third and the fourth carbon atom from the @ carbon, they are called @-3 fatty acids. If the first double bond is between the sixth and seventh carbon atom, they are called @-6 fatty acids. PUFAs are further metabolized in the body by the addition of carbon atoms and by desaturation (extraction of hydrogen). Linoleic acid, which is a @-6 fatty acid, is metabolized to y-linolenic acid, dihomo-γ-linolinic acid, arachidonic acid, adrenic acid, tetracosatetraenoic acid, tetracosapentaenoic acid and docosapentaenoic acid. α-linolenic acid, which is a @-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na K-APTase pump and the fatty acids with increasing degrees of cis-unsaturation had higher potencies. See, Pomposiello, S. I. et al., Hypertension 31:615-20 (1998), which is incorporated by reference herein. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Feddersen, C. O. et al., J. Appl. Physiol. 68 (5): 1799-808 (1990); and see, Spannhake, E. W., et al., J. Appl. Physiol. 44:397-495 (1978) and Wicks, T. C. et al., Circ. Res. 38:167-71 (1976), each of which is incorporated by reference herein.

Many studies have reported effects of EPA and DHA on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F, et al., Hypertension 21:22-8 (1993), and Tagawa, H. et al., J Cardiovasc Pharmacol 33:633-40 (1999), each of which is incorporated by reference herein. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference herein. Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50 (4): 164-70 (2007), which is incorporated by reference herein.

The adrenergic receptors (or adrenoceptors) are a class of G protein-coupled receptors that are a target of catecholamines, especially norepinephrine (noradrenaline) and epinephrine (adrenaline). Epinephrine (adrenaline) interacts with both α- and β-adrenoceptors, causing vasoconstriction and vasodilation, respectively. Although a receptors are less sensitive to epinephrine, when activated, they override the vasodilation mediated by β-adrenoceptors because there are more peripheral al receptors than B-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The al-adrenoreceptor is known for smooth muscle contraction, mydriasis, vasoconstriction in the skin, mucosa and abdominal vicera and sphincter contraction of the gastrointestinal (GI) tract and urinary bladder. The al-adrenergic receptors are member of the Gq protein-coupled receptor superfamily. Upon activation, a heterotrimeric G protein, Gq, activates phospholipase C (PLC). The mechanism of action involves interaction with calcium channels and changing the calcium content in a cell. For review, see Smith R. S. et al., Journal of Neurophysiology 102 (2): 1103-14 (2009), which is incorporated by reference herein. Many cells possess these receptors.

al-adrenergic receptors can be a main receptor for fatty acids. For example, saw palmetto extract (SPE), widely used for the treatment of benign prostatic hyperplasia (BPH), has been reported to bind al-adrenergic, muscarinic and 1,4-dihydropyridine (1,4-DHP) calcium channel antagonist receptors. See, Abe M., et al., Biol. Pharm. Bull. 32 (4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to al-adrenergic, muscarinic and 1,4-DHP calcium channel antagonist receptors.

In certain embodiments, a permeation enhancer can be an adrenergic receptor interacter. An adrenergic receptor interacter refers to a compound or substance that modifies and/or otherwise alters the action of an adrenergic receptor. For example, an adrenergic receptor interacter can prevent stimulation of the receptor by increasing, or decreasing their ability to bind. Such interacters can be provided in either short-acting or long-acting forms. Certain short-acting interacters can work quickly, but their effects last only a few hours. Certain long-acting interacters can take longer to work, but their effects can last longer. The interacter can be selected and/or designed based on, e.g., on one or more of the desired delivery and dose, active pharmaceutical ingredient, permeation modifier, permeation enhancer, matrix, and the condition being treated. An adrenergic receptor interacter can be an adrenergic receptor blocker. The adrenergic receptor interacter can be a terpene (e.g. volatile unsaturated hydrocarbons found in the essential oils of plants, derived from units of isoprenes) or a C3-C22 alcohol or acid, preferably a C7-C18 alcohol or acid. In certain embodiments, the adrenergic receptor interacter can include an aromatic compound, for example, an aromatic alcohol. In certain embodiments, the aromatic alcohol can be a benzyl alcohol. In certain circumstances, the benzyl alcohol can be a substituted benzyl alcohol. The substituted benzyl alcohol can be a C1-C4 substituted phenyl methanol, ethanol, or propanol. For example, the substituted benzyl alcohol can be 2-(4-methylphenyl) propan-2-ol, or cherry propanol. In certain embodiments, the adrenergic receptor interacter can include farnesol, linoleic acid, arachidonic acid, docosahexanoic acid, eicosapentanoic acid, and/or docosapentanoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

The C3-C22 alcohol or acid can be an alcohol or acid having a straight C3-C22 hydrocarbon chain, for example a C3-C22 hydrocarbon chain optionally containing at least one double bond, at least one triple bond, or at least one double bond and one triple bond; said hydrocarbon chain being optionally substituted with C1-4 alkyl, C2-4 alkenyl, C2-4 alkynyl, C1-4 alkoxy, hydroxyl, halo, amino, nitro, cyano, C3-5 cycloalkyl, 3-5 membered heterocycloalkyl, monocyclic aryl, 5-6 membered heteroaryl, C1-4 alkylcarbonyloxy, C1-4 alkyloxycarbonyl, C1-4 alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl. In certain embodiments, the alcohol or acid forming an ester can be a C3-C22 or C3-C16 group.

Fatty acids with a higher degree of unsaturation are effective candidates to enhance the permeation of drugs. Unsaturated fatty acids showed higher enhancement than saturated fatty acids, and the enhancement increased with the number of double bonds. See, A. Mittal, et al. Status of Fatty Acids as Skin Penetration Enhancers-A Review, Current Drug Delivery, 2009, 6, pp. 274-279, which is incorporated by reference herein. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than a fatty acid which has double bond at an odd number position. Cis-unsaturation in the chain can tend to increase activity.

An adrenergic receptor interacter can be a terpene. Hypotensive activity of terpenes in essential oils has been reported. See, Menezes I. A. et al., Z. Naturforsch. 65c: 652-66 (2010), which is incorporated by reference herein. In certain embodiments, the permeation enhancer can be a sesquiterpene. Sesquiterpenes are a class of terpenes that consist of three isoprene units and have the empirical formula C15H24. Like monoterpenes, sesquiterpenes may be acyclic or contain rings, including many unique combinations. Biochemical modifications such as oxidation or rearrangement produce the related sesquiterpenoids. In certain embodiments, the adrenergic receptor interactor can be an active drug moiety, or a derivative or prodrug thereof, for example, an ester.

An adrenergic receptor interacter can be an unsaturated fatty acid such as linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in perfumes and flavors. It has been reported that farnesol selectively kills acute myeloid leukemia blasts and leukemic cell lines in preference to primary hemopoietic cells. See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both Farnesol and N-acetyl-S-trans, trans-farnesyl-L-cysteine (AFC), a synthetic mimic of the carboxyl terminus of farnesylated proteins inhibited vasoconstriction in rat aortic rings.

In certain embodiments, an interacter can be an aporphine alkaloid. For example, an interacter can be a dicentrine.

In an embodiment, the pharmaceutical film can include at least one adrenergic receptor interacter. In certain embodiments, more than one adrenergic receptor interacters can be combined. In certain embodiments, two or three different adrenergic receptor interacters can be combined. A combination of adrenergic receptor interacters can modulate, alter or improve the pharmaceutical film's absorption profiles and organoleptic properties.

In general, an interacter can also be a vasodilator or a therapeutic vasodilator. Vasodilators are drugs that open or widen blood vessels. They are typically used to treat hypertension, heart failure and angina, but can be used to treat other conditions as well, including glaucoma for example. Some vasodilators that act primarily on resistance vessels (arterial dilators) are used for hypertension, and heart failure, and angina; however, reflex cardiac stimulation makes some arterial dilators unsuitable for angina. Venous dilators are very effective for angina, and sometimes used for heart failure, but are not used as primary therapy for hypertension. Vasodilator drugs can be mixed (or balanced) vasodilators in that they dilate both arteries and veins and therefore can have wide application in hypertension, heart failure and angina. Some vasodilators, because of their mechanism of action, also have other important actions that can in some cases enhance their therapeutic utility or provide some additional therapeutic benefit. For example, some calcium channel blockers not only dilate blood vessels, but also depress cardiac mechanical and electrical function, which can enhance their antihypertensive actions and confer additional therapeutic benefit such as blocking arrhythmias.

Vasodilator drugs can be classified based on their site of action (arterial versus venous) or by mechanism of action. Some drugs primarily dilate resistance vessels (arterial dilators; e.g., hydralazine), while others primarily affect venous capacitance vessels (venous dilators; e.g., nitroglycerine). Many vasodilator drugs have mixed arterial and venous dilator properties (mixed dilators; e.g., alpha-adrenoceptor antagonists, angiotensin converting enzyme inhibitors), such as phentolamine.

It is more common, however, to classify vasodilator drugs based on their primary mechanism of action. The figure to the right depicts important mechanistic classes of vasodilator drugs. These classes of drugs, as well as other classes that produce vasodilation, include: alpha-adrenoceptor antagonists (alpha-blockers); Angiotensin converting enzyme (ACE) inhibitors; Angiotensin receptor blockers (ARBs); beta2-adrenoceptor agonists (B2-agonists); calcium-channel blockers (CCBs); centrally acting sympatholytics; direct acting vasodilators; endothelin receptor antagonists; ganglionic blockers; nitrodilators; phosphodiesterase inhibitors; potassium-channel openers; renin inhibitors.

In general, the active or inactive components or ingredients can be substances or compounds that create an increased blood flow or flushing of the tissue to enable a modification or difference (increase or decrease) in transmucosal uptake of the API(s), and/or have a positive or negative heat of solution which are used as aids to modify (increase or decrease) transmucosal uptake.

The arrangement, order, or sequence of penetration enhancer(s) and active pharmaceutical ingredient(s) (API(s)) delivered to the desired mucosal surface can vary in order to deliver a desired pharmacokinetic profile. For example, one can apply the permeation enhancer(s) first by a film, by swab, spray, gel, rinse or by a first layer of a film then apply the API(s) by single film, by swab, or by a second layer of a film. The sequence can be reversed or modified, for example, by applying the API(s) first by film, by swab, or by a first layer of a film, and then applying the permeation enhancer(s) by a film, by swab, spray, gel, rinse or by a second layer of a film. In another embodiment, one may apply a permeation enhancer(s) by a film, and a drug by a different film. For example, the permeation enhancer(s) film positioned under a film containing the API(s), or the film containing the API(s) positioned under a film containing the permeation enhancer(s), depending on the desired pharmacokinetic profile.

For example, the penetration enhancer(s) can be used as a pretreatment alone or in combination with at least one API to precondition the mucosa for further absorption of the API(s). The treatment can be followed by another treatment with neat penetration enhancer(s) to follow the at least one API mucosal application. The pretreatment can be applied as a separate treatment (film, gel, solution, swab etc.) or as a layer within a multilayered film construction of one or more layers. Similarly, the pretreatment may be contained within a distinct domain of a single film, designed to dissolve and release to the mucosa prior to release of the secondary domains with or without penetration enhancer(s) or API(s). The active ingredient may then be delivered from a second treatment, alone or in combination with additional penetration enhancer(s). There may also be a tertiary treatment or domain that delivers additional penetration enhancer(s) and/or at least one API(s) or prodrug(s), either at a different ratio relative to each other or relative to the overall loading of the other treatments. This allows a custom pharmacokinetic profile to be obtained. In this way, the product may have single or multiple domains, with penetration enhancer(s) and API(s) that can vary in mucosal application order, composition, concentration, or overall loading that leads to the desired absorption amounts and/or rates that achieve the intended pharmacokinetic profile and/or pharmacodynamic effect.

The film format can be oriented such that no distinct sides, or such that the film has at least one side of a multiple layer film where the edges are co-terminus (having or meeting at a shared border or limit).

The pharmaceutical composition can be a chewable or gelatin based dosage form, spray, gum, gel, cream, tablet, liquid or film. The composition can include textures, for example, at the surface, such as microneedles or micro-protrusions. Recently, the use of micron-scale needles in increasing skin permeability has been shown to significantly increase transdermal delivery, including and especially for macromolecules. Most drug delivery studies have emphasized solid microneedles, which have been shown to increase skin permeability to a broad range of molecules and nanoparticles in vitro. In vivo studies have demonstrated delivery of oligonucleotides, reduction of blood glucose level by insulin, and induction of immune responses from protein and DNA vaccines. For such studies, needle arrays have been used to pierce holes into skin to increase transport by diffusion or iontophoresis or as drug carriers that release drug into the skin from a microneedle surface coating. Hollow microneedles have also been developed and shown to microinject insulin to diabetic rats. To address practical applications of microneedles, the ratio of microneedle fracture force to skin insertion force (i.e. margin of safety) was found to be optimal for needles with small tip radius and large wall thickness. Microneedles inserted into the skin of human subjects were reported as painless. Together, these results suggest that microneedles represent a promising technology to deliver therapeutic compounds into the skin for a range of possible applications. Using the tools of the microelectronics industry, microneedles have been fabricated with a range of sizes, shapes and materials. Microneedles can be, for example, polymeric, microscopic needles that deliver encapsulated drugs in a minimally invasive manner, but other suitable materials can be used.

Applicants have found that microneedles could be used to enhance the delivery of drugs through the oral mucosa, particularly with the claimed compositions. The microneedles create micron sized pores in the oral mucosa which can enhance the delivery of drugs across the mucosa. Solid, hollow or dissolving microneedles can be fabricated out of suitable materials including, but not limited to, metal, polymer, glass and ceramics. The microfabrication process can include photolithography, silicon etching, laser cutting, metal electroplating, metal electro polishing and molding. Microneedles could be solid which is used to pretreat the tissue and are removed before applying the film. The drug loaded polymer film described in this application can be used as the matrix material of the microneedles itself. These films can have microneedles or micro protrusions fabricated on their surface which will dissolve after forming microchannels in the mucosa through which drugs can permeate.

The term “film” can include films and sheets, in any shape, including rectangular, square, or other desired shape. A film can be any desired thickness and size. In preferred embodiments, a film can have a thickness and size such that it can be administered to a user, for example, placed into the oral cavity of the user. A film can have a relatively thin thickness of from about 0.0025 mm to about 0.250 mm, or a film can have a somewhat thicker thickness of from about 0.250 mm to about 1.0 mm. For some films, the thickness may be even larger, i.e., greater than about 1.0 mm or thinner, i.e., less than about 0.0025 mm. A film can be a single layer or a film can be multi-layered, including laminated or multiple cast films. A permeation enhancer and pharmaceutically active component can be combined in a single layer, each contained in separate layers, or can each be otherwise contained in discrete regions of the same dosage form. In certain embodiments, the pharmaceutically active component contained in the polymeric matrix can be dispersed in the matrix. In certain embodiments, the permeation enhancer being contained in the polymeric matrix can be dispersed in the matrix.

Oral dissolving films can fall into three main classes: fast dissolving, moderate dissolving and slow dissolving. Oral dissolving films can also include a combination of any of the above categories. Fast dissolving films can dissolve in about 1 second to about 30 seconds in the mouth, including more than 1 second, more than 5 seconds, more than 10 seconds, more than 20 seconds, and less than 30 seconds. Moderate dissolving films can dissolve in about 1 to about 30 minutes in the mouth including more than 1 minute, more than 5 minutes, more than 10 minutes, more than 20 minutes or less than 30 minutes, and slow dissolving films can dissolve in more than 30 minutes in the mouth. As a general trend, fast dissolving films can include (or consist of) low molecular weight hydrophilic polymers (e.g., polymers having a molecular weight between about 1,000 to 9,000 daltons, or polymers having a molecular weight up to 200,000 daltons). In contrast, slow dissolving films generally include high molecular weight polymers (e.g., having a molecular weight in millions). Moderate dissolving films can tend to fall in between the fast and slow dissolving films.

It can be preferable to use films that are moderate dissolving films. Moderate dissolving films can dissolve rather quickly, but also have a good level of mucoadhesion. Moderate dissolving films can also be flexible, quickly wettable, and are typically non-irritating to the user. Such moderate dissolving films can provide a quick enough dissolution rate, most desirably between about 1 minute and about 20 minutes, while providing an acceptable mucoadhesion level such that the film is not easily removable once it is placed in the oral cavity of the user. This can ensure delivery of a pharmaceutically active component to a user.

The absorption of the active pharmaceutical ingredient through the mucosa can be governed by several factors including the ability of the molecule to permeate and traverse the oral mucosa to reach the systemic or vascular system. Despite significant improvements to the permeation rate, through the incorporation of a penetration enhancer into the formulation, only a portion of the active pharmaceutical ingredient is delivered transmucosally. The remainder of the active pharmaceutical ingredient is washed away through salivary flow or swallowed and drains into the other mucosal areas such as the pharyngeal, esophogeal and gastrointestinal tract where it is absorbed into the body, for example, by a transpharyngeal, transesophogeal or transgastrointestinal mechanism. The combination of one or more of these absorption routes allows a composition such as a spray to deliver faster the active pharmaceutical ingredient (e.g. epinephrine) blood levels than can be achieved orally alone but due to the high oral bioavailability, also ensures a complete dose is delivered upon every application. The swallowed portion of the active pharmaceutical ingredient however is also exposed to variations in the absorption rate due to, e.g., a food effect during oral administration.

The pharmaceutical composition can be formulated to deliver the active pharmaceutical ingredient via at least a gastrointestinal route. The active pharmaceutical ingredient can also be delivered via at least a pharyngeal route. Such delivery includes the part of the throat behind the mouth and nasal cavity, and above the esophagus and trachea (the tubes going down to the stomach and the lungs). This mode of delivery can result in rapid drug uptake. In certain embodiments, the active pharmaceutical ingredient is delivered via at least a esophageal route. This delivery includes the esophagus, a muscular tube connecting the throat (pharynx) with the stomach. Dosage forms that adhere to the esophageal mucosa and prolong contact have been investigated to improve the efficacy of locally acting agents. These modes of delivery can also result in rapid drug uptake, a large surface area for solute transport, improved drug bioavailability, and noninvasive manner of administering the drug.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug. For example, the pharmaceutically active component can be an ester of epinephrine, for example, dipivefrin. See, e.g., J. Anderson, et al., Site of ocular hydrolysis of a prodrug, dipivefrin, and a comparison of its ocular metabolism with that of the parent compounds, epinephrine, Invest., Ophthalmol. Vis. Sci. July 1980. The time it takes to convert 50% of the prodrug by an enzyme or multiple enzymes to yield neat epinephrine systemically in humans is referred to as the half-life. In certain embodiments, the half-life is less than 1 minute.

In certain embodiments, the composition including a prodrug includes more than one prodrug with each prodrug being a derivative of a pharmaceutically active ingredient. In some of these embodiments, one of the prodrugs is dipivefrin.

In certain embodiments, the first prodrug is a first ester of epinephrine and the second prodrug is a second ester of epinephrine, the first ester of epinephrine and the second ester of epinephrine being different.

In certain embodiments, the prodrug is a compound of formula (I), wherein

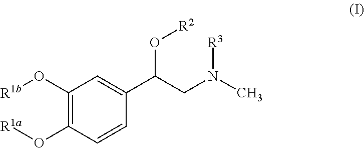

each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof.

In certain embodiments, R² and R³ are H and each R^{1a} and R^{1b}, independently, can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, both of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, one of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

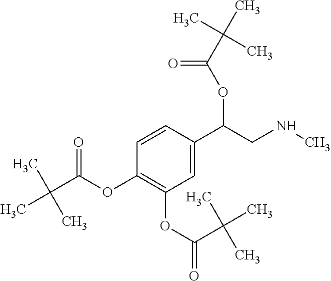 [SMILES: CNCC(OC(=O)C(C)(C)C)c1ccc(OC(=O)C(C)(C)C)c(OC(=O)C(C)(C)C)c1]

AQEP-01

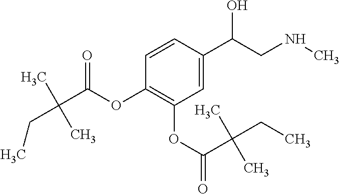 [SMILES: CCC(C)(C)C(=O)Oc1ccc(C(O)CNC)cc1OC(=O)C(C)(C)CC]

AQEP-02

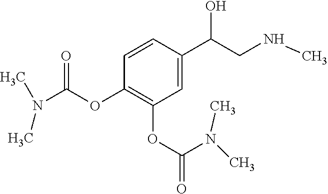

AQEP-03

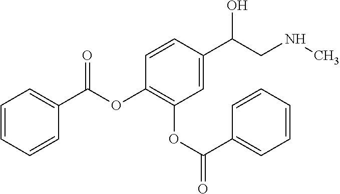 [SMILES: CNCC(O)c1ccc(OC(=O)c2ccccc2)c(OC(=O)c2ccccc2)c1]

AQEP-04

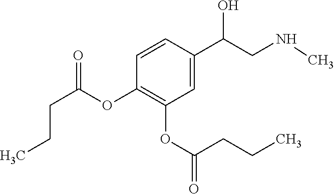 [SMILES: CCCC(=O)Oc1ccc(C(O)CNC)cc1OC(=O)CCC]

AQEP-05

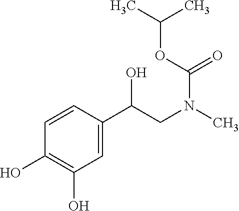 [SMILES: CC(C)OC(=O)N(C)CC(O)c1ccc(O)c(O)c1]

AQEP-06

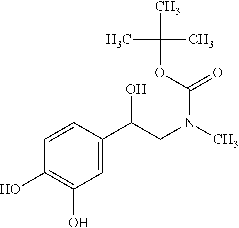 [SMILES: CN(CC(O)c1ccc(O)c(O)c1)C(=O)OC(C)(C)C]

AQEP-07

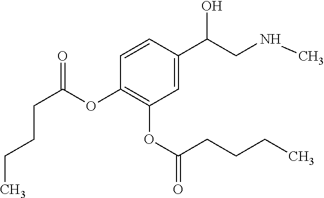 [SMILES: CCCCC(=O)Oc1ccc(C(O)CNC)cc1OC(=O)CCCC]

AQEP-08

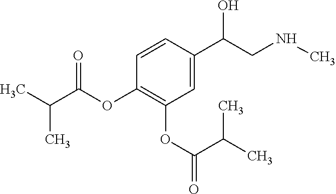 [SMILES: CNCC(O)c1ccc(OC(=O)C(C)C)c(OC(=O)C(C)C)c1]

AQEP-09

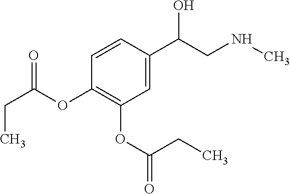 [SMILES: CCC(=O)Oc1ccc(C(O)CNC)cc1OC(=O)CC]

AQEP-10

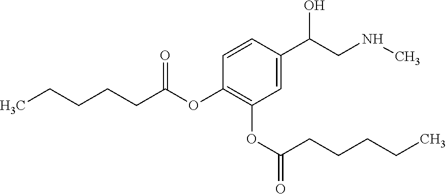 [SMILES: CCCCCC(=O)Oc1ccc(C(O)CNC)cc1OC(=O)CCCCC]

AQEP-11

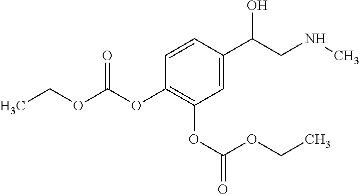 [SMILES: CCOC(=O)Oc1ccc(C(O)CNC)cc1OC(=O)OCC]

AQEP-12

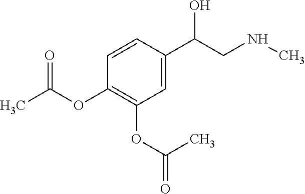 [SMILES: CNCC(O)c1ccc(OC(C)=O)c(OC(C)=O)c1]

AQEP-13 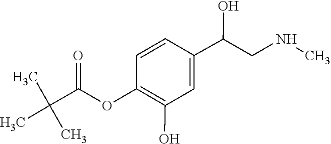 [SMILES: CNCC(O)c1ccc(OC(=O)C(C)(C)C)c(O)c1]
4-Pivaloylepinephrine 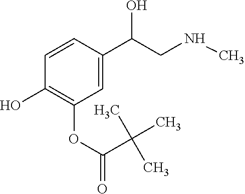 [SMILES: CNCC(O)c1ccc(O)c(OC(=O)C(C)(C)C)c1]
3-Pivaloylepinephrine
AQEP-14 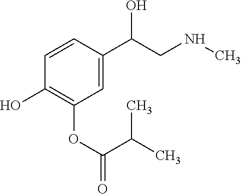 [SMILES: CNCC(O)c1ccc(O)c(OC(=O)C(C)C)c1]
3-isobutyryl epinephrine 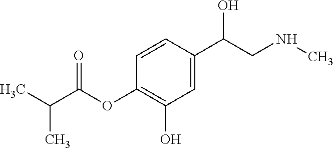 [SMILES: CNCC(O)c1ccc(OC(=O)C(C)C)c(O)c1]
4-isobutyryl epinephrine
AQEP-15

Administering epinephrine as a prodrug such as dipivefrin, or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15 confer certain advantages. For one, dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 are lipophilic and therefore has a higher permeation through a mucosa. Dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 each have a longer plasma half-life due to higher protein binding. Dipivefrin is capable of sustained blood levels, and has a reduced interaction with a-receptors, therefore 10 minimizing or eliminating unwanted or harmful vasoconstriction. Prodrugs, for example, AQEP-09, can exhibit higher binding affinity for a-and-receptors, with binding and activation profiles that are more similar to epinephrine than dipivefrin. Other prodrugs, and combinations of prodrugs, can exhibit binding affinities for α- and β-receptors that favor one or more receptor, similar to or different from epinephrine.

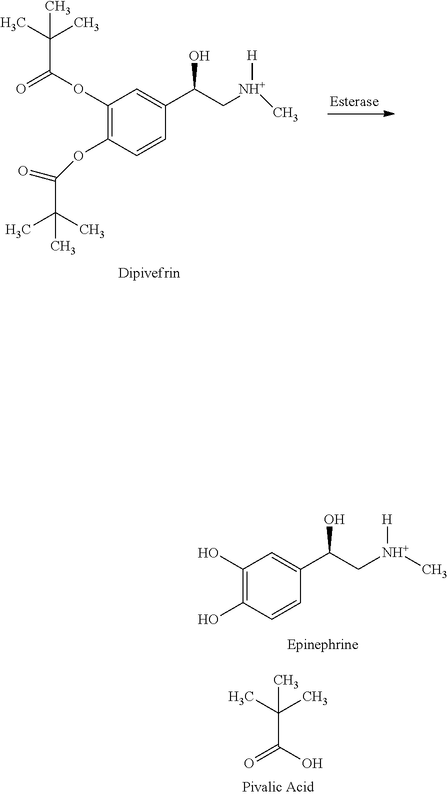

Dipivefrin or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15, alone or in combination, can be delivered in sublingual film in a similar manner as with epinephrine delivered by other methods, including injection.

The compound of formula I can be a pharmaceutically acceptable salt. The pharmaceutically acceptable salt can be an acid addition salt or a base addition salt. Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric ac-id, sulfuric acid and perchloric acid, or with organic acids such as acetic acid, oxalic acid, maleic acid, tartaric acid, citric acid, succinic acid or malonic acid. Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N⁺ (C1-4alkyl) 4 salts. The alkyl can be a hydroxyalkyl. Other pharmaceutically acceptable salts of the compound can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

To deliver epinephrine, a class of prodrug compounds was tested having modifications made to the R^{1a}, R^{1b}, R² and R³ groups of epinephrine as shown below. The R^{1a} and R^{1b} groups can include esters, amides, carbonates and carbamates, orthoesters or acetals. The groups can include for example, alkyl esters, chloroalkyl esters, amides, alkyl amides, chloroalkyl amides. The R² groups can include benzylic alcohol modification. The R³ group can include amine modification or oxazolidines. An ideal prodrug would have one or more of the following attributes, is biologically acceptable, penetrates a tissue, is stable and converts in the body, tissue or blood. In some cases, the prodrug may not need any permeation enhancers at all but rather permeate sufficiently by itself. The conversion of the prodrug to active is not predictable based on chain length of the R^{1a}, R^{1b}, R² and R³ groups. In particular, a tertiary group at the second atom of the R^{1a}, R^{1b}, R² or R³ group. The permeation of the prodrug is also unpredictable based on the R^{1a}, R^{1b}, R² and R³ groups.

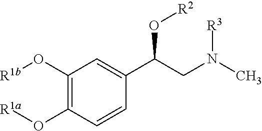

In general, a method of treating a medical condition can include administering a prodrug from a matrix, the prodrug being converted at controlled rate, for example, at a rate of 20 pg/ml to about 40 ng/ml of active compound. The prodrug can be converted at a rate where the active compound cannot be detected in plasma. For example, the prodrug conversion to active compound can be slow and sustained. In certain circumstances, the prodrug conversion to active can be less than 240 minutes, less than 180 minutes, less than 120 minutes, less than 60 minutes, or less than 30 minutes. In other circumstances, the prodrug conversion can be a slow conversion, for example, providing active compound exposure for 4 hours, 6 hours, 8 hours, 12 hours, 18 hours, 24 hours, or longer. For example, the composition can supply active compound for daily, 24 hour dosing.

In certain circumstances, the composition can be administered using microneedles or nanofibers. This type of administration can regulate delivery rates of the prodrug. In other circumstances, this type of administration can be used to direct the prodrug to specific structures in the skin tissue or other tissues.

The prodrug can be converted to 200 pg/ml to about 1200 pg/ml of active compound in less than 120 minutes. In certain embodiments, prodrug is converted to 200 pg/ml to about 1200 pg/ml of active compound in less than 100 minutes. The prodrug can also be converted to 200 pg/ml to about 600 pg/ml of active compound in less than 60 minutes. In certain embodiments, the prodrug is converted to 200 pg/ml to about 600 pg/ml of active compound in less than 45 minutes. In certain embodiments, the prodrug is converted to 200 pg/ml to about 600 pg/ml of active compound in less than 30 minutes.

In certain embodiments, the prodrug converts to create a sustained concentration of 200 pg/ml to about 600 pg/ml of active compound.

In certain embodiments, less than 100% of the prodrug is converted. In other embodiments, 100% of the prodrug is converted.

In general, a method of treating a medical condition comprising administering a prodrug, the prodrug being converted to produce a concentration of active from 20 pg/ml to about 40 ng/ml of active compound in less than 240 minutes and in which 100% of prodrug is converted. In certain circumstances, a method of treating a medical condition comprising administering a prodrug from a matrix, the prodrug being converted to produce a concentration of active from 20 pg/ml to about 40 ng/ml of active compound in less than 240 minutes and in which less than 100% of prodrug is converted. The prodrug can be administered from a matrix.

In certain embodiments, the prodrug can produce therapeutic levels over 100 pg/ml of epinephrine for a duration of at least 1 hour. In certain embodiments, the prodrug can produce therapeutic levels over 100 pg/ml of epinephrine for a duration of at least 2 hours. In certain embodiments, the prodrug produces therapeutic levels over 100 pg/ml of epinephrine for a duration of at least 3 hours. In certain embodiments, the prodrug produces therapeutic levels over 100/ml pg of epinephrine for a duration of at least 4 hours.

A prodrug can be metabolized, for example by hydrolysis. Metabolism can occur through enzymatic conversion, for example through hydrolytic enzymes, which convert a prodrug into an active compound. A prodrug can be converted at various times and in various ways in the body. A prodrug can be designed based on a targeted approach for in any suitable manner based on where and when conversion is desired. In some instances, prodrug conversion can occur systemically (e.g. in circulation). In some situations, prodrug conversion occurs intracellularly (e.g., antiviral nucleoside analogs, lipid-lowering statins). In some situations, prodrug conversion can occur extracellularly, for examples in digestive fluids or other extracellular body fluids).

In certain embodiments, at least half of the administered prodrug is converted in less than 240 minutes. In certain embodiments, at least half of the administered prodrug is converted in less than 120 minutes. In other embodiments, at least half of the administered prodrug is converted in less than 60 minutes. In other embodiments, at least half of the administered prodrug is converted in less than 30 minutes. In other embodiments, at least half of the administered prodrug is converted in less than 15 minutes. In other embodiments, at least half of the administered prodrug is converted in less than 10 minutes. In other embodiments, at least half of the administered prodrug is converted in less than 5 minutes. In other embodiments, at least half of the administered prodrug is converted in less than 1 minute.

For example, when the prodrug is an ester of epinephrine, the half-life of hydrolysis of the ester to form epinephrine in-vivo in a human subject can be less than one minute. For example, a review of pharmacokinetic data from a study in which 12 mg of an ester of epinephrine is delivered by a sublingual film as described herein, epinephrine plasma levels clearly indicate significant systemic absorption of epinephrine, however, the requisite parent prodrug concentration was undetectable. The absence of detectable prodrug in the plasma indicates a conversion half life of the prodrug that is not able to be calculated by traditional pharmacokinetic techniques. The detection limit of the prodrug was 2 pg/ml. By inferring the time between blood sampling timepoints (5 minutes) around the observed Cmax, it can be reasonably concluded that the half life must be less than 1 minute and is very likely to be less than 30 seconds based on the data, in order to adequately clear the requisite prodrug level before the next timepoint.

The half-life of hydrolysis of a prodrug or an ester of epinephrine in-vivo in a human subject should not be confused with the release or dissolution of epinephrine hydrochloride from a film into 500 mL of simulated saliva as described in Alayoubi et al. Pharm. Dev. Technol. 2017, 22 (8), 1012-1016, which is incorporated by reference herein. Note also that the Alayoubi report did not investigate the ability of a film to deliver epinephrine in vivo and did not involve a prodrug or the hydrolysis of any prodrug systemically.

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

For example, the pharmaceutically active component can be buprenorphine, naloxone, acetaminophen, riluzole, clobazam, Rizatriptan, propofol, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, hydrocortisone, predonisolone, dexarnethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, fluocinonide, edaravone, lurasidone, esomeprazole, lumateperone, naldmedine, doxylamine, pyridoxine, diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride dibucaine hydrochloride, dibucaine, lidocaine hydrochloride, lidocaine, benzocaine, p-buthylaminobenzoic acid 2-(die-ethylamino)ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, thimerosal, phenol, thymol, benzalkonium chloride, benzethonium chloride, chlorhexidine, povidone iodide, cetylpyridinium chloride, eugenol, trimethylammonium bromide, naphazoline nitrate, tetrahydrozoline hydrochloride, oxymetazoline hydrochloride, phenylephrine hydrochloride, tramazoline hydrochloride, thrombin, phytonadione, protamine sulfate, aminocaproic acid, tranexamic acid, carbazochrome, carbaxochrome sodium sulfanate, rutin, hesperidin, sulfamine, sulfathiazole, sulfadiazine, homosulfamine, sulfisoxazole, sulfisomidine, sulfamethizole, nitrofurazone, penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, salicylic acid, podophyllum resin, podolifox, cantharidin, chloroacetic acids, silver nitrate, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir, heparin, insulin, LHRH, TRH, interferons, oligonuclides, calcitonin, octreotide, omeprazone, fluoxetine, ethinylestradiol, amiodipine, paroxetine, enalapril, lisinopril, leuprolide, prevastatin, lovastatin, norethindrone, risperidone, olanzapine, albuterol, hydrochlorothiazide, pseudoephridrine, warfarin, terazosin, cisapride, ipratropium, busprione, methylphenidate, levothyroxine, zolpidem, levonorgestrel, glyburide, benazepril, medroxyprogesterone, clonazepam, ondansetron, losartan, quinapril, nitroglycerin, midazolam versed, cetirizine, doxazosin, glipizide, vaccine hepatitis B, salmeterol, sumatriptan, triamcinolone acetonide, goserelin, beclomethasone, granisteron, desogestrel, alprazolam, estradiol, nicotine, interferon beta 1A, cromolyn, fosinopril, digoxin, fluticasone, bisoprolol, calcitril, captorpril, butorphanol, clonidine, premarin, testosterone, sumatriptan, clotrimazole, bisacodyl, dextromethorphan, nitroglycerine, nafarelin, dinoprostone, nicotine, bisacodyl, goserelin, and granisetron. In certain embodiments, the pharmaceutically active component can be epinephrine, a prodrug of epinephrine a benzodiazepine such as diazepam or lorazepam or alprazolam.

In one example, a composition including epinephrine or its salts or esters (such as dipivefrin) can have a biodelivery profile similar to that of epinephrine administered by injection, for example, using an EpiPen. Epinephrine or its prodrug can be present in an amount of from about 0.01 mg to about 100 mg per dosage, for example, at a 0.1 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. In another example, a composition including diazepam can have a biodelivery profile similar to that of a diazepam tablet or gel, or better.

Dipivefrin can be present in an amount of from about 0.5 mg to about 100 mg per dosage, for example, at a 0.5 mg, 1 mg, 5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof.

In another example, a composition (e.g., including epinephrine) can have a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; or (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject. The formulation can include approximately the same active pharmaceutical ingredient (API): enhancer ratio as in the other examples for epinephrine.

Administering epinephrine as a prodrug such as dipivefrin confers certain advantages. For one, dipivefrin is lipophilic and therefore has a higher permeation through a mucosa. It also has a longer plasma half-life due to higher protein binding. It is capable of sustained blood levels, and does not interact with a-receptors, therefore minimizing or eliminating unwanted or harmful vasoconstriction.

A film and/or its components can be water-soluble, water swellable or water-insoluble. The term “water-soluble” can refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water.

The composition can include a polymeric matrix. Any desired polymeric matrix may be used, provided that it is orally dissolvable or erodible. The dosage should have enough bioadhesion to not be easily removed and it should form a gel like structure when administered. They can be moderate-dissolving in the oral cavity and particularly suitable for delivery of pharmaceutically active components, although both fast release, delayed release, controlled release and sustained release compositions are also among the various embodiments contemplated.

The pharmaceutical composition film can include dendritic polymers which can include highly branched macromolecules with various structural architectures. The dendritic polymers can include dendrimers, dendronised polymers (dendrigrafted polymers), linear dendritic hybrids, multi-arm star polymers, or hyperbranched polymers.

Hyperbranched polymers are highly branched polymers with imperfections in their structure. However they can be synthesized in a single step reaction which can be an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications. See, e.g., Dendrimers as Drug Carriers: Applications in Different Routes of Drug Administration. J Pharm Sci, VOL. 97, 2008, 123-143, which is incorporated by reference herein.

The dendritic polymers can have internal cavities which can encapsulate drugs. The steric hindrance caused by the highly dense polymer chains might prevent the crystallization of the drugs. Thus, branched polymers can provide additional advantages in formulating crystallizable drugs in a polymer matrix.

Examples of suitable dendritic polymers include poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers or hyperbranched polymers.

Other examples of hyperbranched polymers include poly(amines) s, polycarbonates, poly(ether ketone) s, polyurethanes, polycarbosilanes, polysiloxanes, poly(ester amine) s, poly(sulfone amine) s, poly(urea urethane) s and polyether polyols such as polyglycerols.

A film can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through a controlled drying processes, which include application of heat and/or radiation energy to the wet film matrix to form a visco-elastic structure, thereby controlling the uniformity of content of the film. The controlled drying processes can include air alone, heat alone or heat and air together contacting the top of the film or bottom of the film or the substrate supporting the cast or deposited or extruded film or contacting more than one surface at the same time or at different times during the drying process. Some of such processes are described in more detail in U.S. Pat. Nos. 8,765,167 and 8,652,378, each of which are incorporated by reference herein. Alternatively, the films may be extruded as described in U.S. Patent Publication No. 2005/0037055 A1, which is incorporated by reference herein.

A polymer included in the films may be water-soluble, water-swellable, water-insoluble, or a combination of one or more either water-soluble, water-swellable or water-insoluble polymers. The polymer may include cellulose, cellulose derivatives or gums. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, films formed from such water-soluble polymers may be sufficiently water-soluble to be dissolvable upon contact with bodily fluids.

Other polymers useful for incorporation into the films include biodegradable polymers, copolymers, block polymers or combinations thereof. It is understood that the term “biodegradable” is intended to include materials that chemically degrade, as opposed to materials that physically break apart (i.e., bioerodable materials). The polymers incorporated in the films can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, polyhydroxy-alkanoates or mixtures thereof. The polymer matrix can include one, two, three, four or more components.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of pharmaceutically active component contained in the composition. Some pharmaceutically active components may only require a few minutes for delivery through the mucosal tissue, whereas other pharmaceutically active components may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods of time, such as up to several hours, which may be desirable for delivery of certain pharmaceutically active components.

Desirably, an individual film dosage of the pharmaceutical film can have a suitable thickness, and small size, which is between about 0.0625-3 inch by about 0.0625-3 inch. The film size can also be greater than 0.0625 inch, greater than 0.5 inch, greater than 1 inch, greater than 2 inches, about 3 inches, and greater than 3 inches, less than 3 inches, less than 2 inches, less than 1 inch, less than 0.5 inch, less than 0.0625 inch in at least one aspect, or greater than 0.0625 inch, greater than 0.5 inch, greater than 1 inch, greater than 2 inches, or greater than 3 inches, about 3 inches, less than 3 inches, less than 2 inches, less than 1 inch, less than 0.5 inch, less than 0.0625 inch in another aspect. The aspect ratio, including thickness, length, and width can be optimized by a person of ordinary skill in the art based on the chemical and physical properties of the polymeric matrix, the active pharmaceutical ingredient, dosage, enhancer, and other additives involved as well as the dimensions of the desired dispensing unit. The film dosage should have good adhesion when placed in the buccal cavity or in the sublingual region of the user. Further, the film dosage should disperse and dissolve at a moderate rate, most desirably dispersing within about 1 minute and dissolving within about 3 minutes. In some embodiments, the film dosage may be capable of dispersing and dissolving at a rate of between about 1 to about 30 minutes, for example, about 1 to about 20 minutes, or more than 1 minute, more than 5 minutes, more than 7 minutes, more than 10 minutes, more than 12 minutes, more than 15 minutes, more than 20 minutes, more than 30 minutes, about 30 minutes, or less than 30 minutes, less than 20 minutes, less than 15 minutes, less than 12 minutes, less than 10 minutes, less than 7 minutes, less than 5 minutes, or less than 1 minute. Sublingual dispersion rates may be shorter than buccal dispersion rates.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight, including greater than 5%, greater than 10%, greater than 15%, greater than 20%, greater than 30%, greater than 40%, greater than 50%, greater than 60%, and greater than 70%, about 70%, less than 70%, less than 60%, less than 50%, less than 40%, less than 30%, less than 20%, less than 10% or less than 5% by weight.

Additives may be included in the films. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, desiccants, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). As used herein, the term “stabilizer” means an excipient capable of preventing aggregation or other physical degradation, as well as chemical degradation, of the active pharmaceutical ingredient, another excipient, or the combination thereof.

Stabilizers may also be classified as plasticizers, desiccants, antioxidants, sequestrants, pH modifiers, emulsifiers and/or surfactants, and UV stabilizers.

An absorbant component, adsorbant component, or desiccant is a compound that is designed to safeguard pharmaceutical products from liquid damage, for example, water or other liquids, by absorbing moisture by physical adsorption or by chemical reaction, thereby improving the stability of the product. A suitable the absorbant component, the adsorbant component, or desiccant can include a silica, fumed silica or a mesoporous silica. The absorbant component, adsorbant component, or desiccant can be an amorphouse silicon dioxide. Examples of the absorbant component, adsorbant component, or desiccant include silicon dioxide variants such as a Cab-o-Sil or a Syloid product. The desiccant can be 1-15% by weight of the pharmaceutical composition. For example, the absorbant component, adsorbant component, or desiccant can be 2%, 3%, 4%, 5%, 6%, 7%, 8%, 9%, 10%, 11%, 12%, 13%, 14%, or 15% w/w of the pharmaceutical composition. The silicon dioxide can have an average surface area of about 25 m²/g, 50 m²/g, 100 m²/g, 200 m²/g, 300 m²/g, 400 m²/g, 500 m²/g, or 600 m²/g. The silicon dioxide can have an average particle size of about 1 micron, 2 microns, 4 microns, 5 microns, 8 microns, about 10 microns or greater. In certain embodiments, one or more oil components of the composition can be adsorbed on the desiccant. The absorbant component, adsorbant component, or desiccant can adsorb up to about 0.5 mL, 1.0 mL, 1.5 mL, or 2.0 mL of liquid per gram of desiccant.

Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, a-tocopherol and the esters thereof, butylated hydroxytoluene and butylated hydroxyanisole. The terms “tocopherol” also includes esters of tocopherol. A known tocopherol is a-tocopherol. The term “a-tocopherol” includes esters of a-tocopherol (for example, a-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more a-D-mannopyranose units linked at the 1,4 positions by a linkages), cyclogalactins (5 or more β-D-galactopyranose units linked at the 1,4 positions by β linkages), cycloaltrins (5 or more a-D-altropyranose units linked at the 1,4 positions by a linkages), and combinations thereof.

pH modifiers include acids (e.g., tartaric acid, citric acid, lactic acid, fumaric acid, phosphoric acid, ascorbic acid, acetic acid, succininc acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C10-C20)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, a-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C10-C24)-alkyl-dimethylammonium chloride or bromide), (C10-C24)-alkyl-dimethylethylammonium chloride or bromide, (C10-C24)-alkyl-trimethylammonium chloride or bromide (e.g., cetyltrimethylammonium chloride or bromide), (C10-C24)-alkyl-dimethylbenzylammonium chloride or bromide (e.g., (C12-C18)-alkyl-dimethylbenzylammonium chloride), N—(C10-C18)-alkyl-pyridinium chloride or bromide (e.g., N—(C12-C16)-alkyl-pyridinium chloride or bromide), N—(C10-C18)-alkyl-isoquinolinium chloride, bromide or monoalkyl sulfate, N—(C12-C18)-alkyl-polyoylaminoformylmethylpyridinium chloride, N—(C12-C18)-alkyl-N-methylmorpholinium chloride, bromide or monoalkyl sulfate, N—(C12-C18)-alkyl-N-ethylmorpholinium chloride, bromide or monoalkyl sulfate, (C16-C18)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, 0-phenanthroline, and citrate. “Detergents” include, but are not limited to, SDS and sodium lauroyl sarcosyl. “Chaotropes” include, but are not limited to guandinium hydrochloride, isothiocyanate, urea, and formamide. As discussed herein, stabilizers can be present in 0.0001%-50% by weight, including greater than 0.0001%, greater than 0.001%, greater than 0.01%, greater than 0.1%, greater than 1%, greater than 5%, greater than 10%, greater than 20%, greater than 30%, greater than 40%, greater than 50%, less than 50%, less than 40%, less than 30%, less than 20%, less than 10%, less than 1%, less than 0.1%, less than 0.01%, less than 0.001%, or less than 0.0001% by weight.

Useful additives can include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, pea starch, pregelatinized hydroxypropyl pea starch, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

The additional components can range up to about 80%, desirably about 0.005% to 50% and more desirably within the range of 1% to 20% based on the weight of all composition components, including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, or less than 1%. Other additives can include anti-tacking, flow agents and opacifiers, such as the oxides of magnesium aluminum, silicon, titanium, etc. desirably in a concentration range of about 0.005% to about 5% by weight and desirably about 0.02% to about 2% based on the weight of all film components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, or less than 0.02%.

In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols sorbitol, sodium diethylsulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition including greater than 0.5%, greater than 1%, greater than 1.5%, greater than 2%, greater than 4%, greater than 5%, greater than 10%, greater than 15%, about 20%, greater than 20%, less than 20%, less than 15%, less than 10%, less than 5%, less than 4%, less than 2%, less than 1%, and less than 0.5%. There may further be added compounds to improve the texture properties of the film material such as animal or vegetable fats, desirably in their hydrogenated form. The composition can also include compounds to improve the textural properties of the product. Other ingredients can include binders which contribute to the ease of formation and general quality of the films. Non-limiting examples of binders include starches, natural gums, pregelatinized starches, gelatin, polyvinylpyrrolidone, methylcellulose, sodium carboxymethylcellulose, ethylcellulose, polyacrylamides, polyvinyloxoazolidone, or polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform films. Agents or components which perform this function can be referred to as densification or densifying agents. As described above, entrapped air or undesired components may lead to non-uniform films.

Any other optional components described in commonly assigned U.S. Pat. Nos. 7,425,292 and 8,765,167, referred to above, also may be included in the films described herein.

The film compositions further desirably contains a buffer so as to control the pH of the film composition. Any desired level of buffer may be incorporated into the film composition so as to provide the desired pH level encountered as the pharmaceutically active component is released from the composition. The buffer is preferably provided in an amount sufficient to control the release from the film and/or the absorption into the body of the pharmaceutically active component. In some embodiments, the buffer may include sodium citrate, citric acid, bitartrate salt and combinations thereof.

The pharmaceutical films described herein may be formed via any desired process. Suitable processes are set forth in U.S. Pat. Nos. 8,652,378, 7,425,292 and 7,357,891, each of which are incorporated by reference herein. In one embodiment, the film dosage composition is formed by first preparing a wet composition, the wet composition including a polymeric carrier matrix and a therapeutically effective amount of a pharmaceutically active component. The wet composition is cast into a film and then sufficiently dried to form a self-supporting film composition. The wet composition may be cast into individual dosages, or it may be cast into a sheet, where the sheet is then cut into individual dosages.

The pharmaceutical composition can adhere to a mucosal surface. The present invention finds particular use in the localized treatment of body tissues, diseases, or wounds which may have moist surfaces and which are susceptible to bodily fluids, such as the mouth, the vagina, organs, or other types of mucosal surfaces. The composition carries a pharmaceutical, and upon application and adherence to the mucosal surface, offers a layer of protection and delivers the pharmaceutical to the treatment site, the surrounding tissues, and other bodily fluids. The composition provides an appropriate residence time for effective drug delivery at the treatment site, given the control of erosion in aqueous solution or bodily fluids such as saliva, and the slow, natural erosion of the film concomitant or subsequent to the delivery.

The residence time of the composition depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water soluble organic and inorganic salts; by using crosslinking agents such as glyoxal with polymers such as hydroxyethyl cellulose for partial crosslinking; or by post-treatment irradiation or curing, which may alter the physical state of the film, including its crystallinity or phase transition, once obtained. These strategies might be employed alone or in combination in order to modify the erosion kinetics of the film. Upon application, the pharmaceutical composition film adheres to the mucosal surface and is held in place. Water absorption softens the composition, thereby diminishing the foreign body sensation. As the composition rests on the mucosal surface, delivery of the drug occurs. Residence times may be adjusted over a wide range depending upon the desired timing of the delivery of the chosen pharmaceutical and the desired lifespan of the carrier. Generally, however, the residence time is modulated between about a few seconds to about a few days. Preferably, the residence time for most pharmaceuticals is adjusted from about 5 seconds to about 24 hours. More preferably, the residence time is adjusted from about 5 seconds to about 30 minutes. In addition to providing drug delivery, once the composition adheres to the mucosal surface, it also provides protection to the treatment site, acting as an erodable bandage. Lipophilic agents can be designed to slow down erodability to decrease disintegration and dissolution.

It is also possible to adjust the kinetics of erodability of the composition by adding excipients which are sensitive to enzymes such as amylase, very soluble in water such as water soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the composition.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, allyl ethers, alkyl aryl ethers, sorbitan monostearate, sorbitan monooleate and/or sorbitan monopalmitate.

The amount of pharmaceutically active component to be used depends on the desired treatment strength and the composition of the layers, although preferably, the pharmaceutical component comprises from about 0.001% to about 99%, more preferably from about 0.003 to about 75%, and most preferably from about 0.005% to about 50% by weight of the composition, including, more than 0.005%, more than 0.05%, more than 0.5%, more than 1%, more than 5%, more than 10%, more than 15%, more than 20%, more than 30%, about 50%, more than 50%, less than 50%, less than 30%, less than 20%, less than 15%, less than 10%, less than 5%, less than 1%, less than 0.5%, less than 0.05%, or less than 0.005%. The amounts of other components may vary depending on the drug or other components but typically these components comprise no more than 50%, preferably no more than 30%, and most preferably no more than 15% by total weight of the composition.

The thickness of the film may vary, depending on the thickness of each of the layers and the number of layers. As stated above, both the thickness and amount of layers may be adjusted in order to vary the erosion kinetics. Preferably, if the composition has only two layers, the thickness ranges from 0.005 mm to 2 mm, preferably from 0.01 to 1 mm, and more preferably from 0.1 to 0.5 mm, including greater than 0.1 mm, greater than 0.2 mm, about 0.5 mm, greater than 0.5 mm, less than 0.5 mm, less than 0.2 mm, or less than 0.1 mm. The thickness of each layer may vary from 10 to 90% of the overall thickness of the layered composition, and preferably varies from 30 to 60%, including greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, or less than 10%. Thus, the preferred thickness of each layer may vary from 0.01 mm to 0.9 mm, or from 0.03 to 0.5 mm.

As one skilled in the art will appreciate, when systemic delivery, e.g., transmucosal or transdermal delivery is desired, the treatment site may include any area in which the film is capable of delivery and/or maintaining a desired level of pharmaceutical in the blood, lymph, or other bodily fluid. Typically, such treatment sites include the oral, esophageal, aural, ocular, anal, nasal, and vaginal mucosal tissue, as well as, the skin. If the skin is to be employed as the treatment site, then usually larger areas of the skin wherein movement will not disrupt the adhesion of the film, such as the upper arm or thigh, are preferred.

Methods of administering a pharmaceutical composition are described, for example, in U.S. application Ser. No. 18/778,562 which is incorporated by reference herein.

The pharmaceutical composition can also be used as a wound dressing. By offering a physical, compatible, oxygen and moisture permeable, flexible barrier which can be washed away, the film can not only protect a wound but also deliver a pharmaceutical in order to promote healing, aseptic, scarification, to ease the pain or to improve globally the condition of the sufferer. Some of the: examples given below are well suited for an application to the skin or a wound. As one skilled in the art will appreciate, the formulation might require incorporating a specific hydrophilic/hygroscopic excipient which would help in maintaining good adhesion on dry skin over an extended period of time. Another advantage of the present invention when utilized in this manner is that if one does not wish that the film be noticeable on the skin, then no dyes or colored substances need be used. If, on the other hand, one desires that the film be noticeable, a dye or colored substance may be employed.

The following examples illustrate certain embodiments of the claimed methods and compositions.

### EXAMPLES

### Example 1

In one embodiment, an epinephrine pharmaceutical composition film can be made with the following formulation:

| | Formulation A | | |
|---|---|---|---|
| MATERIAL | WT % dry | WT % wet | mg/Strip |
| EPINEPHRINE bitartrate | 46.40 | 18.56 | 54.56 |
| hydroxypropylmethyl cellulose | 11.54 | 4.61 | 13.57 |
| polyvinyl pyrrolidone | 27.92 | 11.17 | 32.84 |
| Glycerol monooleate | 0.58 | 0.23 | 0.68 |
| Polyethylene Oxide | 1.16 | 0.46 | 1.36 |
| Polysorbate | 0.58 | 0.23 | 0.68 |
| Phytoextract | 9.98 | 3.99 | 9.97 |
| Stabilizer | 0.12 | 0.05 | 0.14 |
| Buffer | 0.58 | 0.23 | 0.68 |
| Artificial sweetener | 1.16 | 0.46 | 1.36 |
| Linoleic acid | 0.0037 | 0.00 | 0.00 |
| Farnesol | | | |
| Yellow # 5 | | | |
| TOTAL | 100.00 | 40.00 | 115.84 |

### Example 2

An exemplary epinephrine pharmaceutical film composition was made with the following formulation:

| | Formulation B | | |
|---|---|---|---|
| MATERIAL | WT % dry | WT % wet | mg/Strip |
| EPINEPHRINE bitartrate | 46.17 | 18.47 | 54.29 |
| Hydroxypropylmethyl cellulose | 11.48 | 4.59 | 13.50 |
| Polyvinyl pyrrolidone | 27.78 | 11.11 | 32.67 |
| Glycerol monooleate | 0.58 | 0.23 | 0.68 |
| Polyethylene Oxide | 1.15 | 0.46 | 1.35 |
| Polysorbate | 0.58 | 0.23 | 0.68 |
| Phytoextract | 9.93 | 3.97 | 9.92 |
| Stabilizer | 0.12 | 0.05 | 0.14 |
| Buffer | 0.58 | 0.23 | 0.68 |
| Artificial sweetener | 1.15 | 0.46 | 1.35 |
| Linoleic acid | 0.50 | 0.20 | 0.59 |
| Farnesol | | | |
| Yellow # 5 | | | |
| TOTAL | 100.00 | 40.00 | 115.85 |

### Example 3

In another embodiment, pharmaceutical film compositions were made with the following formulation:

| | Formulation C | | |
|---|---|---|---|
| MATERIAL | WT % dry | WT % wet | mg/Strip |
| EPINEPHRINE bitartrate | 46.35 | 18.54 | 54.51 |
| Hydroxypropylmethyl cellulose | 11.53 | 4.61 | 13.55 |
| Polyvinyl pyrrolidone | 27.90 | 11.16 | 32.80 |
| Glycerol monooleate | 0.58 | 0.23 | 0.68 |
| Polyethylene oxide | 1.16 | 0.46 | 1.36 |
| Polysorbate | 0.58 | 0.23 | 0.68 |
| Phytoextract | 9.97 | 3.99 | 9.96 |
| Stabilizer | 0.12 | 0.05 | 0.14 |
| Buffer | 0.58 | 0.23 | 0.68 |
| Artificial sweetener | 1.16 | 0.46 | 1.36 |
| Linoleic acid | | | |
| Farnesol | 0.10 | 0.04 | 0.06 |
| Yellow # 5 | | | |
| TOTAL | 100.00 | 40.00 | 115.78 |

### Example 4

In another embodiment, pharmaceutical film compositions were made with the following formulation:

| | Formulation D | | |
|---|---|---|---|
| MATERIAL | WT % dry | WT % wet | mg/Strip |
| EPINEPHRINE bitartrate | 46.07 | 18.43 | 54.52 |
| Hydroxypropylmethyl cellulose | 11.46 | 4.58 | 13.56 |
| Polyvinyl pyrrolidone | 27.73 | 11.09 | 32.81 |
| Glycerol monooleate | 0.57 | 0.23 | 0.68 |
| Polyethylene oxide | 1.15 | 0.46 | 1.36 |
| Polysorbate | 0.57 | 0.23 | 0.68 |
| Phytoextract | 9.91 | 3.96 | 9.96 |
| Stabilizer | 0.11 | 0.05 | 0.14 |
| Buffer | 0.57 | 0.23 | 0.68 |
| Artificial sweetener | 1.15 | 0.46 | 1.36 |
| Linoleic acid | 0.10 | 0.04 | 0.06 |
| Farnesol | 0.50 | 0.20 | 0.29 |
| Yellow # 5 | 0.10 | 0.04 | 0.06 |
| TOTAL | 100.00 | 40.00 | 116.16 |

### Example 5

In one embodiment, a dipivefrin pharmaceutical composition film can be made with the following formulation:

| MATERIAL | WT % dry | mg/Strip |
|---|---|---|
| Dipivefrin | 3.45 | 3.297 |
| HPMC E15 | 23.22 | 22.165 |
| PVP K90 | 57.57 | 54.959 |
| GMO | 0.50 | 0.476 |
| PEO N10 | 0.50 | 0.476 |
| PEG400 | 9.25 | 8.830 |
| clove oil | 3.00 | 2.437 |
| sodium citrate | 0.50 | 0.476 |
| sucralose | 1.00 | 0.952 |
| labrasol | 1.00 | 0.956 |
| green #3 | 0.01 | 0.010 |
| TOTAL | 100.00 | 95.03 |

### Example 6

In another embodiment, a dipivefrin pharmaceutical composition film can be made with the following formulation:

| MATERIAL | WT % dry | mg/Strip |
|---|---|---|
| Dipivefrin | 6.00 | 6.602 |
| HPMC | 21.22 | 23.350 |
| PVP | 51.52 | 56.690 |
| GMO | 0.50 | 0.548 |
| PEO | 0.50 | 0.548 |
| PEG400 | 9.25 | 10.179 |
| clove oil | 3.00 | 2.809 |
| citric acid | 3.00 | 3.301 |
| sodium citrate | 2.00 | 2.201 |
| sodium metabisulfite | 1.00 | 1.100 |
| sucralose | 1.00 | 1.097 |
| labrasol | 1.00 | 1.102 |
| green #3 | 0.01 | 0.011 |
| TOTAL | 100.00 | 109.54 |

### Example 7

In another embodiment, a dipivefrin pharmaceutical composition film can be made with the following formulation:

| MATERIAL | WT % dry | mg/Strip |
|---|---|---|
| Dipivefrin | 10.50 | 13.204 |
| HPMC | 20.22 | 25.427 |
| PVP | 48.02 | 60.385 |
| GMO | 0.50 | 0.627 |
| PEO | 0.50 | 0.627 |
| PEG400 | 9.25 | 11.633 |
| clove oil | 3.00 | 3.211 |
| citric acid | 3.00 | 3.773 |
| sodium citrate | 2.00 | 2.515 |
| sodium metabisulfite | 1.00 | 1.258 |
| sucralose | 1.00 | 1.254 |
| labrasol | 1.00 | 1.259 |
| green #3 | 0.01 | 0.013 |
| TOTAL | 100.00 | 125.18 |

### Example 8

In an embodiment, a diisobutyryl epinephrine (DESF) dry film composition can have the following formulation. In an embodiment, the pharmaceutical film can include at least one adrenergic receptor interacter (e.g., DESF F5). In certain embodiments, more than one adrenergic receptor interacters can be added (e.g., DESF F10). In certain embodiments, two or three different adrenergic receptors for example, can be combined to modulate, alter or improve the pharmaceutical film's absorption profiles and organoleptic properties (e.g., DESF Formulations 284-1-1, 285-1-1 and 286-1-1):

| Material | Dry Film % w/w |
|---|---|
| Diisobutyryl epinephrine HCl | 25.00-30.00 |
| Pregelatinized hydroxypropyl pea starch | 1.000-19.000 |
| Polyvinylpyrrolidone | 21.000-24.000 |
| Polyethylene oxide | 1.000 |
| Silicon dioxide | 5.000-11.600 |
| Silicon dioxide (second) | 5.000-11.600 |
| Glycerol monooleate | 1.000 |
| Polysaccharide | 1.000 |
| 8-Hydroxy-p-cymene | 3.333-7.500 |
| Benzyl alcohol | 3.333-15.000 |
| Eugenol | 3.333-17.500 |
| Hydrochloric acid | 0-0.667 |
| Sweetener | 1.500-1.990 |
| Polacrilex resin | 1.000 |
| Sodium fluoride | 0.700 |
| Sugar alcohol | 0-0.500 |
| Edetate disodium, dihydrate | 1.000 |
| Surfactant | 1.000 |
| Mint flavor | 1.000 |
| Mixed berry flavor | 7.000-9.000 |
| Monoammonium glycyrrhizinate | 0-0.250 |
| Glycyrrhizic acid | 0-0.030 |
| FD&C Color | 0.010 |

Ester prodrugs can be prone to hydrolysis and loss of assay after storage was attributed to the presence of residual moisture in the films. In order to minimize the presence of moisture (free water) in this example, an absorbant component such as silicon dioxide was used. The absorbant component was found to reduce the hydrolysis degradation and result in a films with reduced tackiness.

### Example 9—Oral Allergy Study

The Oral Allergy Syndrome (OAS) challenge study was designed as a two-part investigation to evaluate the pharmacokinetics (PK) and pharmacodynamics (PD) of Anaphylm™ (epinephrine) in adults with allergen-induced oral physiological change.

Part 1 enrolled thirty-five subjects (n=35) with confirmed OAS into a three-period study with the following arms: (1) Anaphylm with allergen exposure, (2) Anaphylm without allergen exposure (control) and (3) Adrenalin intramuscular (IM) injection without allergen exposure (control). Part 2 was an optional follow-on to Part 1 where subjects received the alternate dosing regimen (either single or repeat) not administered in Part 1, with allergen challenge; a single control arm (Adrenalin) was also included. During allergen exposure arms in Parts 1 and 2, subjects were exposed to a fruit they were known to be allergic to, and the resulting symptoms were documented for location, severity, and duration. Similar assessments on subjects were performed during the allergen exposure immediately prior to the dosing of Anaphylm and symptom resolution was compared to that of the baseline reaction from screening that provided no treatment. During study conduct, there were no reports of difficulty administering Anaphylm to subjects.

Following allergen exposure, all subjects reported symptoms consistent with their known allergies. Approximately thirty-five percent of subjects reported swelling of their lips, cheeks, and/or throat. Additional symptoms included tingling, pain, nasal congestion, and other symptoms associated with edema. Ninety-two percent of subjects were categorized as having moderate or severe symptoms according to the pre-defined oral severity score.

In Part A, patients (n=35) were exposed to an allergen prior to dosing to determine the patient profile and determine the symptoms post OAS challenge.

| Allergen | # Exposures |
|---|---|
| Pineapple | 14 |
| Red Apple | 7 |
| Kiwi | 7 |
| Banana | 2 |
| Cherry | 3 |
| Avocado | 2 |
| Dragonfruit | 1 |
| Fig | 2 |
| Grapefruit | 2 |
| Lemon | 1 |
| Lychee | 2 |
| Peach | 1 |
| Strawberry | 1 |
| Tangerine | 2 |
| Total | 47* |

In Part A, 35 patients were studied. In Part B, 12 patients returned, for a total of 47 exposures.

The patient profiles were assessed as follows:

| Patient Profile | Symptoms post OAS challenge | Mucosal Changes |
|---|---|---|
| 17% severe | 100% had Oral symptoms | 100% reported symptoms of allergic |
| 77% moderate | 36% also had systemic | response in mucosa |
| 6% mild | symptoms | 81% reported ≥ 2 symptoms of |
| | | allergic response in mucosa |
| | | 25% reported swelling |

The observed symptoms were as follows:

| | % of subjects at dosing |
|---|---|
| Symptoms of Interest | (n = 47) |
| Lip swelling | 31.9% |
| Throat swelling | 10.6% |
| Tongue swelling | 6.4% |
| Cheek swelling | 4.3% |
| Nasal congestion | 2.1% |
| Sublingual swelling | 2.1% (1) |

The median time for total subject symptom resolution was twelve minutes with Anaphylm. This is faster than the median time to total subject symptom resolution without any intervention which was seventy-four minutes. After Anaphylm administration, individual symptom resolution was observed as early as two minutes and the median time for individual symptom resolution was five minutes.

Anaphylm PK results in subjects exposed to an allergen remained similar to exposure without an allergen and to previous profiles from the Company's pivotal study in healthy subjects The time to maximum plasma concentration, or Tmax, remained at 12 minutes in both the allergen and non-allergen administrations for a single dose of Anaphylm. The maximum plasma concentration, or Cmax, was comparable between Anaphylm administered with and without allergen challenge. In addition, Anaphylm was safe and well-tolerated with all adverse events categorized as mild or moderate and resolving without medical intervention.

Referring to FIG. 1A, this shows the rapid symptom resolution after a single dose and after a repeat oral dose of the pharmaceutical composition. From the challenge start, maximum symptoms were observed within 2-3 minutes, and dosing was performed within 2-5 minutes of maximum symptoms. After treatment of the pharmaceutical composition, the median symptom resolution was 12 minutes for oral symptoms and 10 minutes for body symptoms.

Referring to FIG. 1B, this shows the correlation of symptom relief and plasma epinephrine (in pg/ml) after a single dose. The onset of resolution of all symptoms accelerated with the method of treatment and after administering the pharmaceutical composition

(Anaphylm). The median time to complete symptom resolution was 12 minutes after Anaphylm administration. By contrast, median time to resolution was 74 minutes without Anaphylm administrations. Surprisingly, even after 2 minutes, the number of allergic symptoms began to dramatically decrease to about half the amount: from over 120 to under 60 for a single dose, and from over 160 to under 60 for a repeat dose. After 5 minutes, the number of allergic symptoms measured about 15 for a single dose, and about 25 for a repeat dose. After 10 minutes the number of allergic symptoms measured about 25 for a single dose, and about 30 for a repeat dose. After 15 minutes the number of allergic symptoms measured about 10 for a single dose, and about 20 for a repeat dose. After 20 minutes the number of allergic symptoms measured under 20 for a single dose, and about 15 for a repeat dose. After 30 minutes the number of allergic symptoms measured about 5 for a single dose, and about 5 for a repeat dose.

Referring to FIG. 1C, symptom relief was also observed with repeat dosing.

Referring to FIG. 1D, this shows the pharmacokinetic profile of the oral pharmaceutical composition remained consistent with and without exposure to allergen.

### Cmax and Tmax

| Administration | Cmax (pg/mL) | Median Tmax (min) |
|---|---|---|
| IM (n = 24) | 261.2 | 50 |
| Anaphylm with allergen | 403.5 | 12 |
| (n = 23) | | |
| Anaphylm without allergen | 372.8 | 12 |
| (n = 15) | | |

Partial AUC's (hr*pg/mL)

| | AUC | AUC | AUC | AUC |
|---|---|---|---|---|
| Administration | 0-10 min | 0-20 min | 0-30 min | 0-45 min |
| IM (n = 24) | 6.0 | 18.9 | 39.0 | 76.0 |
| Anaphylm with | 14.4 | 63.2 | 97.0 | 132.1 |
| allergen (n = 23) | | | | |
| Anaphylm without | 11.1 | 50.3 | 82.6 | 124.1 |
| allergen (n = 15) | | | | |

Referring to FIG. 1E, this shows the pharmacokinetic profile of the oral pharmaceutical composition remained consistent with and without exposure to allergen compared to pivotal data.

### Cmax and Tmax

| Administration | Cmax (pg/mL) | Median Tmax (min) |
|---|---|---|
| IM (n = 22) | 538.8 | 57.5 |
| Anaphylm with allergen | 1194.0 | 25 |
| (n = 23) | | |
| Anaphylm without allergen | 585.5 | 25 |
| (n = 13) | | |

Partial AUC's (hr*pg/mL)

| | AUC | AUC | AUC | AUC |
|---|---|---|---|---|
| Administration | 0-10 min | 0-20 min | 0-30 min | 0-45 min |
| IM (n = 22) | 5.1 | 15.5 | 39.2 | 99.4 |
| Anaphylm with | 10.1 | 62.6 | 216.8 | 360.5 |
| allergen (n = 23) | | | | |
| Anaphylm without | 9.2 | 35.0 | 106.5 | 180.4 |
| allergen (n = 13) | | | | |

For both FIG. 1D and FIG. 1E, this shows the geometric mean baseline-adjusted epinephrine concentration over time in OAS subjects after single dose administration and shows that the pharmaceutical film pharmacokinetic profile remains consistent with and without allergen exposure. The data for manual intramuscular (IM) is shown in triangle points. The data from the allergen challenge in shown in square points. The data from no allergen challenge is shown in circle points.

### Example 10—Pharmacodynamics, Blood Pressure and Heart Rate

The pharmaceutical film (Anaphylm) was found to elicit the desired pharmacodynamic response in key metrics of Systolic Blood Pressure (SBP) Diastolic Blood Pressure (DBP) and Pulse (HR), consistent with and without OAS induced oral changes.

Referring to FIG. 2A, this shows change in SBP as a function of time, comparing intramuscular (IM) administration against the pharmaceutical film (Anaphylm) for a single dose.

Referring to FIG. 2B, this shows change in DBP as a function of time, comparing intramuscular (IM) administration against the pharmaceutical film (Anaphylm) for a single dose.

Referring to FIG. 2C, this shows change in heart rate as a function of time, comparing intramuscular (IM) administration against the pharmaceutical film (Anaphylm) for a single dose

Referring to FIG. 3A, this shows change in SBP as a function of time, comparing intramuscular (IM) administration against the pharmaceutical film (Anaphylm) for a repeat dose.

Referring to FIG. 3B, this shows change in DBP as a function of time, comparing intramuscular (IM) administration against the pharmaceutical film (Anaphylm) for a repeat dose.

Referring to FIG. 3C, this shows change in heart rate as a function of time, comparing intramuscular (IM) administration against the pharmaceutical film (Anaphylm) for a single dose. This shows Anaphylm elicits the desired pharmacodynamic response in key metrics of Systolic Blood Pressure (SBP) Diastolic Blood Pressure (DBP) and Pulse (HR), consistent with and without OAS induced oral changes for both single and repeat dosing.

The following symptoms were observed in the safety summary. Most adverse events were categorized as mild. No severe adverse events were observed. Most adverse events were transient and resolved without intervention. The primary cardiovascular events were associated with mild palpitations. No interventions were required.

| | | 12 mg | | |
|---|---|---|---|---|
| | | Anaphylm | 0.3 mg | 12 mg |
| Preferred | | with AC | Man. IM² | Anaphylm |
| Term System | | Incidence (%) | Incidence (%) | Incidence (%) |
| Organ Class | Severity | N = 24 | N = 24 | N = 16 |
| Cardiac | | | | |
| Disorders | | | | |
| Palpitations | Mild | 2 (8.3%) | 0 | 0 |
| (subjective, | | | | |
| patient- | | | | |
| reported) | | | | |
| Gastro- | | | | |
| intestinal | | | | |
| Disorders | | | | |
| Nausea | | 1 (4.2%) | 0 | 0 |

The following OAS is the repeat dose safety summary

| | | 12 mg × 2 | | | 12 mg × 2 |
|---|---|---|---|---|---|
| | | Anaphylm | | | Anaphylm |
| | | with | | | without |
| | | allergen | | 0.3 mg × 2 | allergen |
| | | challenge | | Man. IM | challenge |
| System | | Incidence (%) | | Incidence (%) | Incidence (%) |
| Organ Class | Severity | N = 24 | | N = 23 | N = 16 |
| Cardiac | | | | | |
| Disorders | | | | | |
| Palpitations | Mild | 4 | (16.7%) | 0 | 0 |
| (subjective, | | | | | |
| subject- | | | | | |
| reported) | | | | | |
| Gastro- | | | | | |
| intestinal | | | | | |
| Disorders | | | | | |
| Vomiting | Mild | 1 | (4.2%) | 0 | 1 (6.3%) |
| Nausea | Mild | 2 | (8.3%) | 0 | 0 |

### Example 11—Single and Repeat Doses of Sublingual Film and Intramuscular Injection

The following study was conducted to compare the pharmacokinetics (PK) and pharmacodynamics (PD) of single dose (SD) and repeat doses (RD) AQST-109 and IM epinephrine in healthy adults.

In this two-part, phase 3, open-label study, participants received SD and RD of AQST-109, and epinephrine via manual syringe, EpiPen®, or Auvi-Q® autoinjector on different days. PK and PD parameters were assessed over 360 minutes post-administration; primary endpoints-maximum serum concentration (Cmax) and partial area under the curve (AUC). Additional PK, blood pressure and heart rate were also measured.

Healthy, non-atopic, nonsmoking participants aged 18-55 years were eligible for this open-label study and were screened and recruited. Volunteers who passed the initial screening were required to undergo additional study-specific screening procedures after providing informed consent and before receiving the drug. Participants were required to have a corrected QT interval ≤450 milliseconds, a body mass index ≥18 and ≤30 kg/m², and a weight ≥45 kg for females and ≥50 kg for males. Participants were excluded if they had previous exposure to AQST-109 or epinephrine in the past 30 days, or a known history or presence of hypersensitivity or idiosyncratic reaction to epinephrine or any other drug substances with similar activity.

In the single dose (SD) part of the study, participants were randomized to the order in which they received one dose of all four of the following treatments: AQST-109 (12 mg, Aquestive Pharmaceuticals, USA), epinephrine via manual injection (0.3 mg/0.3 mL, Belcher Pharmaceuticals, LLC, USA), epinephrine via EpiPenR autoinjector (0.3 mg, Mylan Pharmaceuticals, USA), or epinephrine via Auvi-QR autoinjector (0.3 mg, Kaleo Inc., USA), after an overnight fast of ≥10 hours, with a 4-hour post-injection fast. There was a 7-day washout period between administration of dosing formulations.

In the repeat dose (RD) part of the study, participants were randomized to the order in which they received the following three treatments, each of which was given twice (initial dose at time 0, then repeat dose after 15 minutes): AQST-109, epinephrine via manual injection, or epinephrine via EpiPenR autoinjector. This was also performed after an overnight fast of ≥10 hours, with a 4-hour post-injection fast and a 7-day washout period between administration of epinephrine formulations. Of note, the RD study was performed first, followed by the SD, to verify optimization of the AQST-109 dose quantity.

Following SD AQST-109 administration, AUC and Cmax were successfully bracketed beyond 60 minutes, exceeding manual IM injection, equaling EpiPen®, and lower than Auvi-QR. AQST-109 had a median time to maximum concentration (Tmax) of 12 minutes, exceeding EpiPen® (20 minutes), Auvi-Q® (30 minutes), IM injection (50 minutes), and was more consistent (narrower interquartile range) than comparators. AQST-109 demonstrated more rapid and larger increases in blood pressure and heart rate than comparators. RD AQST-109 showed dose-proportional increases in PK and PD parameters, exceeding comparators. No serious adverse events were reported.

Primary endpoints were baseline-corrected epinephrine maximum measured plasma analyte concentration over the sampling period (Cₘₐₓ) and area under the analyte concentration vs time curve (AUC)_{0-10 min}, AUC_{0-20 min}, AUC_{0-30 min}, and AUC_{0-45 min} in the SD part. Secondary endpoints were baseline-corrected epinephrine AUC_{0-10 min}, AUC_{0-20 min}, AUC_{0-30 min}, and concentration at 45 and 60 minutes in the RD part. Exploratory endpoints in both parts included baseline-corrected systolic and diastolic blood pressure and heart rate maximum effect (Eₘₐₓ), time to achieve Eₘₐₓ, and area under the effect curve from 0-60 minutes

Descriptive statistics for baseline-corrected epinephrine for each nominal time point were tabulated for each treatment, including number of observations, mathematic mean, standard deviation, geometric mean, coefficient of variation (CV %), median, minimum, and maximum. In addition, standard error was calculated for baseline-corrected epinephrine. Descriptive statistics for baseline-corrected epinephrine PK parameters were provided for each treatment, including number of observations, mathematic mean, standard deviation, geometric mean, CV %, median, minimum, and maximum. Descriptive statistics for baseline-corrected systolic and diastolic blood pressure and heart rate for each nominal time point were provided for each treatment, including number of observations, mathematic mean, standard deviation, median, 25th percentile, 75th percentile, minimum, and maximum. Descriptive statistics for baseline-corrected PD parameters were provided for each treatment, including number of observations, arithmetic mean, standard deviation, median, minimum, and maximum.

To evaluate the pharmacokinetic biocomparability of AQST-109 versus approved reference-listed drugs (RLDs), a PK “bracketing” strategy was used. First, a biocomparability bracket, defined by the range of PK parameters observed for RLD autoinjectors and manual injections, was established in a prior study. In the present randomized crossover trial, SD PK profiles of AQST-109 were directly compared against those of at least two RLDs whose PK metrics define that bracket. Demonstrating that AQST-109's PK parameters fall within this predefined RLD range creates a direct biocomparability bridge, and the data generated herein serve as the primary evidence of AQST-109's comparability to existing therapies.

### Results

The results show that SD AQST-109 demonstrated consistently bracketed PK and PD responses comparable to IM epinephrine, with more rapid Tmax. These results suggest that AQST-109 represents a promising, highly portable, unobtrusive, and needle-free alternative to currently approved epinephrine formulations. There were 64 participants enrolled in the SD part of the study, with 61 dosed in all four periods and receiving all four study treatments. Two participants discontinued due to voluntary withdrawal from the study, and one participant discontinued due to adverse events (AEs).

There were 36 participants enrolled in the RD part of the study, with 34 participants dosed in all three periods and receiving all three treatments. Two participants discontinued before completing treatment (both due to AEs). Four participants discontinued after completing the RD and did not continue to the SD (two due to AEs, one due to withdrawal by participant, and one due to an abnormal poststudy laboratory result).

Baseline demographic characteristics are reported for the participants from the SD and RD parts (Table 1) and were comparable among treatment groups.

TABLE 1
Baseline demographic characteristics

| | Participants in SD | | Participants in RD | |
|---|---|---|---|---|
| Characteristic | (n = 64) | | (n = 36) | |
| Female, n (%) | 28 | (43.8) | 18 | (50.0) |
| Median age, years | 39 | (19-55) | 40 | (23-55) |
| (range) | | | | |
| Median weight, kg | 74.5 | (52.2-100.5) | 73.1 | (54.9-96.1) |
| (range) | | | | |
| Median height, cm | 169.8 | (152.7-187.7) | 166.9 | (152.7-181.5) |
| (range) | | | | |
| Median BMI, kg/m² | 26.5 | (19-30) | 27.0 | (21-30) |
| (range) | | | | |
| Race, n (%) | | | | |
| White | 33 | (51.6) | 21 | (58.3) |
| Black or African | 21 | (32.8) | 9 | (25.0) |
| American | | | | |
| Asian | 9 | (14.1) | 6 | (16.7) |
| Multiracial^{a} | 1 | (1.6) | 0 | |
| Ethnicity, n (%) | | | | |
| Hispanic or Latino | 15 | (23.4) | 9 | (25.0) |

BMI, body mass index; SD, single dose; RD, repeat dose.
^{a}Black or African American and White.

### Pharmacokinetics

The PK parameters for the SD and RD parts of the study are presented in Table 2 and Table 3.

TABLE 2
Pharmacokinetic parameters: single dose part

| | | | | | Manual ≤ | Manual ≤ |
|---|---|---|---|---|---|---|
| | Treatment | | | | AQST-109 ≤ | AQST-109 ≤ |
| Parameter^{a} | AQST-109 | Manual | EpiPen ® | Auvi-Q ® | EpiPen ® | Auvi-Q ® |
| Cₘₐₓ (pg/mL) | 470.2 | 308.2 | 469.2 | 520.6 | No | Yes |
| AUC_{0-5 min} | 1.563 | 1.026 | 5.237 | 5.134 | Yes | Yes |
| (hr*pg/mL) | | | | | | |
| AUC_{0-10 min} | 17.67 | 3.265 | 23.39 | 21.85 | Yes | Yes |
| (hr*pg/mL) | | | | | | |
| AUC_{0-20 min} | 71.26 | 11.52 | 62.03 | 61.50 | No | No |
| (hr*pg/mL) | | | | | | |
| AUC_{0-30 min} | 104.8 | 29.66 | 109.7 | 112.4 | Yes | Yes |
| (hr*pg/mL) | | | | | | |
| AUC_{0-45 min} | 138.6 | 74.59 | 179.8 | 200.3 | Yes | Yes |
| (hr*pg/mL) | | | | | | |
| AUC_{0-60 min} | 165.0 | 133.8 | 233.9 | 269.0 | Yes | Yes |
| (hr*pg/mL) | | | | | | |
| Tₘₐₓ, median | 12.0 | 55.0 | 25.0 | 30.0 | No | No |
| (minutes) | | | | | | |

AUC, area under the analyte concentration vs time curve; Cₘₐₓ, concentration over the sampling period; Tₘₐₓ, time to peak concentration.
^{a}Geometric mean unless otherwise indicated.

TABLE 3
Pharmacokinetic parameters: repeat dose part.

| | | | | Bracketing | |
|---|---|---|---|---|---|
| | Treatment | | | Manual ≤ | EpiPen ® ≤ |
| Parameter^{a} | AQST-109 | Manual | EpiPen ® | AQST-109 | AQST-109 |
| Cₘₐₓ (pg/mL) | 2159 | 539.4 | 898.9 | Yes | Yes |
| AUC_{0-5 min} | 1.436 | 1.653 | 4.906 | No | No |
| (hr*pg/mL) | | | | | |
| AUC_{0-10 min} | 19.61 | 4.077 | 20.99 | Yes | No |
| (hr*pg/mL) | | | | | |
| AUC_{0-15 min} | 46.17 | 6.520 | 35.70 | Yes | Yes |
| (hr*pg/mL) | | | | | |
| AUC_{0-20 min} | 121.2 | 13.79 | 69.79 | Yes | Yes |
| (hr*pg/mL) | | | | | |
| AUC_{0-25 min} | 269.4 | 24.66 | 118.8 | Yes | Yes |
| (hr*pg/mL) | | | | | |
| AUC_{0-30 min} | 399.7 | 38.96 | 172.3 | Yes | Yes |
| (hr*pg/mL) | | | | | |
| AUC_{0-35 min} | 480.3 | 57.35 | 223.9 | Yes | Yes |
| (hr*pg/mL) | | | | | |
| AUC_{0-45 min} | 578.6 | 110.6 | 319.0 | Yes | Yes |
| (hr*pg/mL) | | | | | |
| AUC_{0-60 min} | 673.5 | 214.0 | 439.4 | Yes | Yes |
| (hr*pg/mL) | | | | | |
| Conc_{45 min} | 409.7 | 364.7 | 507.9 | Yes | No |
| (pg/mL) | | | | | |
| Conc_{60 min} | 299.4 | 431.6 | 354.5 | No | No |
| (pg/mL) | | | | | |
| Tₘₐₓ, median | 25.0 | 55.0 | 25.0 | No | Yes |
| (minutes) | | | | | |

AUC, area under the analyte concentration vs time curve; Cₘₐₓ, concentration over the sampling period; Conc, concentration; Tₘₐₓ, time to peak concentration.
^{a}Geometric mean unless otherwise indicated.

For the SD part, the PK profile of AQST-109 was successfully bracketed between IM epinephrine via manual injection and EpiPenR for SD administration. The epinephrine Cmax for AQST-109 (470.2 pg/mL) was beneath that for Auvi-QR (520.6 pg/mL), equal to EpiPenR (469.2 pg/mL), and exceeded manual injection (308.2 pg/mL).

Referring to FIG. 4, the AQST-109 median Tmax was 12 minutes, which was faster than for EpiPen (20 minutes), Auvi-QR (30 minutes), and manual injection (50 minutes). In addition, the IQR for the Tmax was smaller for AQST-109 than for EpiPenR or Auvi-QR, showing a more consistently rapid effect. Serum area under the curve exceeded comparators over the first 30 minutes, then remained bracketed (above manual injection and beneath both autoinjectors) past 60 minutes. AQST-109 exceeded a serum concentration of 100 pg/mL, a proposed surrogate for physiological activity (equal to approximately 3-fold the physiologic level), within 5 minutes, and sustained levels above this mark for more than 60 minutes.

Referring to FIG. 5A and FIG. 5B, the peak plasma baseline-corrected epinephrine concentration in the SD part was earlier (Tmax) for AQST-109 than manual injection, EpiPenR, or Auvi-QR (FIG. 5A) and was higher (Cmax) after repeat AQST-109 than for repeat manual injection or EpiPenR in the RD part (FIG. 5B).

### Pharmacodynamics

Referring to FIGS. 6A-6D, AQST-109 demonstrated an early and rapid increase in baseline-corrected systolic and diastolic blood pressure compared with all reference treatments in both the SD and RD parts.

In the first 30 minutes after administration, the measured increases were both more rapid and greater with AQST-109, and there was no associated transient decrease in diastolic blood pressure as was demonstrated with all three injectable RLD comparators. Referring to FIGS. 7A and 7B, changes in baseline-corrected heart rate were greater with AQST-109 compared with the injectable forms, with a higher though slightly more delayed peak.

There were no deaths or serious AEs reported in either part of the study. In the SD part, treatment-emergent adverse events (TEAEs) were reported in 63/64 (98.4%) participants who received AQST-109. The most common TEAEs among participants treated with AQST-109 were administration site paresthesia (n=54 [84.4%]), application site exfoliation (loosening of the top epithelia layer, n=44 [68.8%]), administration site discomfort (n=34 [53.1%]), and administration site hypoesthesia (n=27 [42.2%]; Table 4). TEAEs were reported in 10/62 (16.1%) participants who received manual injection, 16/63 (25.4%) participants who received EpiPen®, and 8/61 (13.1%) participants who received Auvi-Q® (Table 4).

In the RD part, TEAEs were reported in all 36 (100%) participants who received AQST-109 (Table 5). The most common TEAEs among participants treated with AQST-109 were administration site paresthesia (n=34 [94.4%]), application site exfoliation (n=22 [61.1%]), administration site erythema (n=19 [52.8%]), and administration site hypoesthesia (n=19 [52.8%]; Table 5). TEAEs were reported in 9/36 (25.0%) who received manual injection, and 8/35 (22.9%) who received EpiPen®; the most common TEAEs in participants treated with manual injection and EpiPen® are listed in Table 6.

TABLE 4
TEAEs in comparators: single dose

| | Participants in single dose part (n = 36) | | | | |
|---|---|---|---|---|---|
| TEAEs in ≥5% of participants | Manual | | EpiPen ® | | Auvi-Q ® |
| after any treatment, n (%) | (n = 62) | | (n = 63) | | (n = 61) |
| General disorders and | 4 | (6.5) | 7 | (11.1) | 3 (4.9) |
| administration site conditions | | | | | |
| Injection site pain | 3 | (4.8) | 7 | (11.1) | 3 (4.9) |
| Nervous system disorders | 7 | (11.3) | 9 | (14.3) | 5 (8.2) |
| Somnolence | 3 | (4.8) | 0 | | 1 (1.6) |
| Headache | 2 | (3.2) | 5 | (7.9) | 2 (3.3) |
| Tremor | 1 | (1.6) | 4 | (6.3) | 2 (3.3) |
| Cardiac disorders | 1 | (1.6) | 5 | (7.9) | 1 (1.6) |
| Palpitations | 1 | (1.6) | 4 | (6.3) | 1 (1.6) |

TEAE, treatment-emergent adverse event.

TABLE 5
Treatment-emergent adverse events (TEAEs) for AQST-109

| | AQST-109 | | AQST-109 | |
|---|---|---|---|---|
| TEAEs in ≥5% of patients after | single dose | | repeat dose | |
| any treatment, n (%) | (n = 64) | | (n = 36) | |
| General disorders and | 62 | (96.9) | 36 | (100) |
| administration site conditions | | | | |
| Administration site paresthesia | 54 | (84.4) | 34 | (94.4) |
| Application site exfoliation | 44 | (68.8) | 22 | (61.1) |
| Administration site discomfort | 34 | (53.1) | 8 | (22.2) |
| Administration site hypoesthesia | 27 | (42.2) | 19 | (52.8) |
| Administration site erythema | 21 | (32.8) | 19 | (52.8) |
| Administration site pallor | 15 | (23.4) | 3 | (8.3) |
| Administration site ulcer | 9 | (14.1) | 8 | (22.2) |
| Administration site pain | 8 | (12.5) | 16 | (44.4) |
| Gastrointestinal disorders | 33 | (51.6) | 21 | (58.3) |
| Abdominal discomfort | 14 | (21.9) | 10 | (27.8) |
| Dyspepsia | 10 | (15.6) | 3 | (8.3) |
| Regurgitation | 6 | (9.4) | 4 | (11.1) |
| Nausea | 6 | (9.4) | 12 | (33.3) |

| | | | |
|---|---|---|---|
| Vomiting | — | 2 | (5.6) |
| Upper abdominal pain | — | 2 | (5.6) |

| | | | | |
|---|---|---|---|---|
| Nervous system disorders | 9 | (14.1) | 8 | (22.2) |
| Headache | 4 | (6.3) | 2 | (5.6) |
| Dizziness | 3 | (4.7) | 2 | (5.6) |

| | | | |
|---|---|---|---|
| Tremor | — | 5 | (13.9) |
| Cardiac disorders | — | 6 | (16.7) |
| Palpitations | — | 5 | (13.9) |
| Vascular disorders | — | 2 | (5.6) |
| Hypertension | — | 2 | (5.6) |

TEAE, treatment-emergent adverse event.

TABLE 6
TEAEs in comparators: repeat dose

| | Participants in repeat dose part (n = 36) | | | |
|---|---|---|---|---|
| TEAEs in ≥5% of participants | Manual | | EpiPen ® | |
| after any treatment, n (%) | (n = 36) | | (n = 35) | |
| General disorders and | 4 | (11.1) | 3 | (8.6) |
| administration site | | | | |
| conditions | | | | |
| Injection site pain | 3 | (8.3) | 1 | (2.9) |
| Nervous system disorders | 8 | (22.2) | 4 | (11.4) |
| Headache | 5 | (13.9) | 2 | (5.7) |
| Cardiac disorders | 1 | (2.8) | 3 | (8.6) |
| Palpitations | 1 | (2.8) | 2 | (5.7) |
| Psychiatric disorders | 2 | (5.6) | 2 | (5.7) |
| Anxiety | 2 | (5.6) | 2 | (5.7) |

TEAE, treatment-emergent adverse event.

The study showed that the AQST-109 PK and PD were comparable with established, FDA-approved RLDs for IM epinephrine injection. Results from the SD part demonstrated that administrations of AQST-109 reliably yielded baseline-corrected epinephrine concentrations that met the bracketing criteria for all primary PK parameters throughout the first hour.

AQST-109 had the fastest Tmax versus all the IM comparators, and the Tmax was more consistent with a narrower IQR of 5 minutes compared to 25 minutes for EpiPen®, and 32 minutes for Auvi-Q® (see, e.g., FIG. 4). AQST-109 demonstrated a Cmax equal to EpiPen® but lower than Auvi-Q® and Tmax faster than any approved or investigational formulation of epinephrine published to date. The PK parameters AUC_{0-10 min}, AUC_{0-30 min}, and AUC_{0-45 min} for baseline-corrected epinephrine all met the bracketing criteria as well. The SD administrations of AQST-109 did not lead to any serious AEs.

Results from RD demonstrated that AQST-109 administered at 0 and 15 minutes yielded more than dose-proportional increases of epinephrine concentrations compared to administration of a single dose and did not demonstrate any safety concerns. The geometric means of the PK parameters AUC_{0-10 min}, AUC_{0-30 min}, and AUC_{0-45 min} for baseline-corrected epinephrine were higher following AQST-109 than manual injection, with the partial AUCs following AQST-109 being greater than repeat IM dosing estimates. The higher exposure was not associated with any serious AEs.

In the SD part of the study, PD performance of AQST-109 showed rapid and higher systolic and diastolic blood pressure and heart rate responses versus comparators, sustained throughout the first hour post-dose. Importantly, no transient decrease in diastolic blood pressure was noted with AQST-109, whereas it was seen with the IM comparators. Avoiding any reduction in diastolic blood pressure during anaphylaxis is a desirable profile for maintaining adequate mean arterial pressure and preserving myocardial ability to optimally perfuse, in particular with a patient suffering hypotension and demonstrating signs of hemodynamic instability. Responses with repeat AQST-109 dosing were proportional and exceeded the IM comparators.

There were no serious adverse events reported in either part of the study. The most frequently reported adverse events (AEs) were administration site AEs, mostly paresthesia and erythema, which are attributable to the eugenol component of AQST-109. The majority were mild in severity, with no severe or serious AEs reported. The rapid resolution of treatment emergent adverse events required minimal or no intervention. Overall, AQST-109 was found to be safe and well tolerated when administered as a single or repeat dose.

### Example 12—Phase 2 Open Label Study

A study was performed to compare the pharmacokinetics (PK) and pharmacodynamics (PD) between AQST-109 after oral allergen challenge (OAC) and epinephrine manual intramuscular (IM) injection in adults with oral allergy syndrome (OAS). This was a Phase 2, two-part, open-label, randomized, fixed-sequence, stratified, crossover study to evaluate PK and PD of epinephrine in adults with OAS, administered as single (12 mg) and repeat doses (12 mg×2) of AQST-109 (with and without OAC) or single (0.3 mg) and repeat doses (0.3 mg×2) of manual IM injection.

There were 36 participants enrolled. All participants had medical documentation of OAS. Participants were 18-55 years of age with a documented history of OAS (confirmed during screening), a body weight ≥50 kg for males or ≥45 kg for females, and a body mass index (BMI) ≥18 kg/m² to ≤32.5 kg/m². All participants had a baseline Oral Mucositis Assessment Score of 0, and no prior history of anaphylaxis. This was a Phase 2, two-part, open-label, randomized, fixed-sequence, stratified crossover study designed to evaluate the PK of epinephrine administered via sublingual AQST-109 or manual IM injection across different conditions and dosing regimens. Moderate or severe symptoms during OAC at screening were reported by 94.4% of

participants. Geometric mean values for all primary and secondary PK parameters (Cmax, AUC_{0-10 min}, AUC_{0-20 min}, AUC_{0-30 min}, AUC_{0-45 min}, AUC_{0-60 min}) were greater with AQST-109 after OAC versus manual IM injection. Oral physiological changes did not diminish the PK performance of AQST-109; single-dose PK curves were similar between conditions with and without OAC. PD parameters remained within the normal physiological range. Almost all participants experienced a mild treatment-emergent adverse event (TEAE), with 4 TEAEs considered moderate in severity.

Oral physiological changes associated with OAS did not negatively impact the PK performance of AQST-109, supporting its reliability in scenarios that mimic real-world allergic reactions. No new safety concerns were identified.

Referring to FIG. 8, this illustrates the Phase 2, two-part, open-label, randomized, fixed-sequence, stratified crossover study designed to evaluate the pharmacokinetics (PK) of epinephrine administered via sublingual AQST-109 or manual IM injection across different conditions and dosing regimens.

### Oral Allergy Challenge (OAC)

During the OAC, participants were challenged with 10 grams of the allergen known to trigger an oral allergic reaction. Participants rubbed the allergen on their upper lip, lower lip, and gums and then placed it on their tongue for up to 15 minutes, with a goal of achieving maximum tolerated symptoms. Saliva could be swallowed, and participants were permitted to chew the allergen; however, swallowing the allergen was not permitted. If accidental swallowing occurred, the allergen was replaced with fresh allergen. Allergen replacement was permitted up to 3 times within the 15-minute exposure window.

Severity of oral symptoms was measured before OAC and continued until symptom resolution using the verbal rating scale (VRS). The VRS included a symptom-level rating scale for each potential oral location (tongue, lips, cheeks, under tongue, and throat), system organ class (SOC; respiratory, nasal, integumentary, gastrointestinal, cardiovascular, neurological, constitutional, and facial). Each symptom is measured separately to get symptom-level severity and duration data. The VRS was also used to determine overall severity classification. The symptoms were scored from 0-3, with 0 indicating no symptoms, 1 indicated mild severity, defined as feeling itching, tingling, swelling, pain/irritation but discomfort was not bothersome, 2 indicated moderate severity, defined as feeling itching, tingling, swelling, pain/irritation and discomfort was bothersome, 3 indicated severe severity, defined as discomfort interrupts daily activities, would typically seek relief. Overall severity was determined as following: none, defined as no discomfort in any oral location, with or without other SOC involvement; mild, defined as ≥1 mild oral symptom, with or without other SOC involvement; moderate, defined as ≥1 moderate oral symptom or >3 mild oral symptoms, with or without another SOC involvement; and severe, defined as ≥1 severe oral symptom, ≥2 moderate oral symptoms and additional SOC involvement, or ≥1 moderate oral symptom and >2 moderate symptoms in ≥2 additional SOC involvement.

Qualifying participants were randomized 1:1 to either the single-dose cohort (12 mg AQST-109 or 0.3 mg epinephrine manual IM injection) or repeat-dose cohort (12 mg×2 AQST-109 or 0.3 mg×2 IM epinephrine). Per-protocol randomization by OAC overall severity was initially planned to ensure 25% of participants in each dosing condition experienced moderate or severe symptoms. This stratification was ultimately not performed because the majority of enrolled participants already presented with moderate to severe symptoms.

### Treatment Periods

Treatment 1 (AQST-109 with Oral Allergen Challenge)

The same OAC procedures used during screening were used in Treatment 1. AQST-109 was administered within 5 minutes following removal of the allergen. In the repeat-dose cohort, a second dose of AQST-109 was administered 15 minutes after the first dose. All participants were observed for 4 hours post-dose to assess safety, PK, and PD parameters.

Treatment 2 (Manual IM Epinephrine without OAC)

Following a minimum 24-hour washout period after Treatment 1, participants received a single or repeat dose (according to their assignment) of epinephrine via manual IM injection without an OAC. As in Treatment 1, participants were monitored for 4 hours after dosing to assess safety, PK, and PD parameters.

Treatment 3 (AQST-109 without OAC)

After a minimum 14-day washout period following Treatment 1, participants received a single or repeat dose (according to their assignment) of AQST-109 without an OAC. Post-dosing observations continued for 4 hours to assess safety, PK, and PD parameters.

In Part 1 participants completed all three treatment periods. In Part 2, a subset of 12 participants from Part 1 (6 from each the single- and repeat-dosing cohort) who agreed to crossover to the alternate dosing cohort and completed Treatments 1 and 2 were dosed. For all treatment periods, participants fasted for at least 2 hours prior to dosing and 4 hours post-dose.

### Endpoints

The primary PK endpoints included maximum plasma concentration (Cmax) and partial area under the curve (AUC) values at 0-10, 0-20, 0-30, and 0-45 minutes post-dose. A primary endpoint was considered met if the geometric mean value for AQST-109 was greater than or equal to that of the manual IM injection, aligning with the objective to demonstrate comparable or superior early absorption to an approved comparator. AQST-109 was deemed to have a comparable PK profile if its Cmax and at least three of the four partial AUCs met or exceeded the corresponding values for manual IM injection.

The secondary PK endpoints included characterizing PK parameters following a single dose of AQST-109 with and without an OAC. These included the AUC from time zero to the time of the last measurable analyte concentration (AUC₀₋ₜ), AUC_{0-5 min}, AUC_{0-60 min}, AUC from time 0 to infinity (AUC_{0-∞}). Cₘₐₓ, time to maximum plasma concentration (Tₘₐₓ), elimination rate constant (Kₑₗ), and half-life (t½). Additionally, the PK profile (Cmax, AUC₀₋ₜ, AUC_{0-∞}) of epinephrine following single and repeat dosing of AQST-109 was assessed. The cumulative proportion of participants reaching a therapeutic concentration of epinephrine (uncorrected epinephrine [not adjusted for baseline endogenous epinephrine levels], ≥100 pg/mL) at 5-, 10-, 15-, 20-, 30-, 45-, and 60-minutes post-dose was evaluated by treatment group.²⁷

The PD endpoints included changes in systolic blood pressure (SBP), diastolic blood pressure (DBP), and heart rate (HR) following AQST-109 administration after OAC. Safety and tolerability were assessed through the monitoring of adverse events (AEs), which were coded using Medical Dictionary for Regulatory Activities (Version 27.0). The severity of AEs was classified as mild, moderate or severe, and the relationship to the study drug was determined by the Investigator. Exploratory endpoints included OAC symptoms and time to symptom resolution.

### Statistical Analysis

Descriptive statistics were used to summarize epinephrine plasma concentrations. Geometric mean values for the primary PK endpoints were calculated for both AQST-109 and manual IM injection. PK data from Part 1 and 2 were pooled by treatment and dosing cohort for analysis. For PD assessments, maximum change from baseline (Eₘₐₓ), time to maximum change (TEₘₐₓ), and area under the effect curve from time zero to 60 minutes (AUEC_{0-60 min}) were calculated for SBP, DBP, and HR, as indicates in FIGS. 10A, 10B, 11A, 11B, 12A and 12B.

### Results

A total of 36 participants were randomized in Part 1, equally divided between the single dose (n=18) and repeat-dose (n=18) cohorts. In those assigned to the single dose, one participant discontinued due to a protocol deviation. In those assigned to the repeat dose, two participants discontinued, one prior to treatment 3 by choice and one due to a treatment-emergent adverse event (TEAE). All the other participants completed all 3 periods. In Part 2, 12 participants (n=6 per cohort) from Part 1 crossed over to the alternate cohort, and all completed the assigned treatments.

Demographics and baseline characteristics are shown in Table 7. The mean (SD) age was 31.8 (8.2) years in Part 1 and 35.1 (8.5) years in Part 2, with a predominance of male participants in both parts (61.1% and 83.3% in Part 1 and Part 2, respectively).

TABLE 7
Baseline demographics and characteristics

| | Single-dose cohort | | Repeat-dose cohort | |
|---|---|---|---|---|
| Parameter | (n = 24) | | (n = 24) | |
| Age, years, mean ± SD | 32.2 ± 8.6 | (18-50) | 33.0 ± 8.1 | (18-50) |
| (range) | | | | |
| Sex, n (%) | | | | |
| Male | 15 | (62.5) | 17 | (70.8) |
| Female | 9 | (37.5) | 7 | (29.2) |
| Race, n (%) | | | | |
| Black/African American | 13 | (54.2) | 13 | (54.2) |
| White | 6 | (25.0) | 7 | (29.2) |
| Asian | 5 | (20.8) | 1 | (4.2) |
| Mixed race | 0 | | 2 | (8.3) |
| American Indian or Alaskan | 0 | | 1 | (4.2) |
| Native | | | | |

| | | |
|---|---|---|
| Weight, kg, mean ± SD | 76.0 ± 15.0 | 79.0 ± 12.9 |
| Height, cm, mean ± SD | 172.2 ± 11.6 | 172.4 ± 10.7 |
| BMI, kg/m², mean ± SD | 25.5 ± 3.4 | 26.6 ± 3.4 |

As shown in Table 8, across both the single- and repeat-dose conditions, AQST-109 demonstrated greater geometric mean values for all primary PK parameters (Cₘₐₓ, AUC_{0-10 min}, AUC_{0-20 min}, AUC_{0-30 min}, and AUC_{0-45 min}) and secondary PK parameter (AUC_{0-60 min}) following OAC compared to manual IM injection without OAC. Median Tmax was faster with AQST-109 (12 min for single dose, 25 min for repeat dose) compared with manual IM injection (50 min for single dose, 57 min for repeat dose) in both conditions. With a single-dose, AQST-109 achieved higher geometric mean values for total exposure (AUC₀₋ₜ and AUC₀₋₃) compared with manual IM injection. In both the single- and repeat-dose conditions, AUC_{0-5 min} was higher with manual IM injection compared with AQST-109. AQST-109 epinephrine plasma levels declined across all regimens by 60 to 90 minutes post-dose.

TABLE 8
Comparison of PK parameters of baseline-corrected epinephrine

| | Single dose | | | | Repeat dose | | | |
|---|---|---|---|---|---|---|---|---|
| | AQST-109 | | Manual IM | | AQST-109 | | Manual IM | |
| | with OAC | | injection | | with OAC | | injection | |
| Parameter | (n = 24) | | (n = 24) | | (n = 24) | | (n = 23) | |
| Cₘₐₓ, pg/mL | 404 | (180) | 261 | (61.6) | 1190 | (145) | 539 | (41.8) |
| AUC_{0-5 min}, h*pg/mL | 0.5 | (3760) | 1.4 | (506 | 0.3 | (19000) | 1.77 | (90.4) |
| AUC_{0-10 min}, h*pg/mL | 14.5 | (175) | 6.2 | (111) | 7.0 | (710) | 5.8 | (112) |
| AUC_{0-20 min} h*pg/mL | 62.7 | (163) | 19.0 | (94.7) | 58.4 | (158) | 17.7 | (95.3) |
| AUC_{0-30 min} h*pg/mL | 98.6 | (136) | 38.8 | (82.2) | 210 | (128) | 39.8 | (101) |
| AUC_{0-45 min} h*pg/mL | 127 | (117) | 72.5 | (81.5) | 352 | (116) | 101 | (90.3) |
| AUC_{0-60 min} h*pg/mL | 160 | (101) | 123 | (83.9) | 436 | (107) | 193 | (78.0) |
| AUC₀₋ₜ h*pg/mL | 306 | (76.8) | 360 | (45.0) | 848 | (83.3) | 672 | (40.9) |
| AUC_{0-∞} h*pg/mL | 425 | (87.2) | 499 | (30.9) | 1510 | (80.1) | 955 | (35.7) |
| Median Tₘₐₓ (min) | 12 | | 50 | | 25 | | 57 | |
| t_{1/2} (h) | 1.5 | (62.6) | 1.2 | (8.6) | 2.0 | (24.9) | 1.2 | (34.7) |
| Kₑₗ (1/h) | 0.5 | (62.6) | 0.6 | (8.6) | 0.4 | (24.9) | 0.6 | (34.7) |
| CL/F (L/h) | 28200 | (87.2) | 601 | (30.9) | 15900 | (80.1) | 628 | (35.7) |
| CL/F/kg (L/h/kg) | 359 | (79.1) | 8.5 | (50.2) | 189 | (63.7) | 7.0 | (29.8) |
| V_{d}/F (L) | 55200 | (65.6) | 1050 | (40.1) | 41000 | (62.7) | 978 | (57.5) |
| V_{d}/F/kg (L/kg) | 702 | (65.9) | 14.9 | (60.3) | 488 | (53.1) | 10.9 | (51.2) |

AUC, area under the curve, AUC₀₋ₜ, is the area under the analyte concentration versus time curve, from time 0 to the time of the last measurable concentration, as calculated by the linear up/log down variant of the trapezoidal method; AUC_{0-∞}, The area under the analyte concentration versus time curve from time 0 to infinity as calculated by AUCₜ+Cₜ/Kₑₗ, where Cₜ is the last measurable analyte concentration; CL/F, total body clearance calculated as dose/AUC_{0-∞}; CL/F/kg, total body clearance normalized by body weight; Cₘₐₓ, maximum measured concentration over the sampling period; IM, intramuscular; Kel, elimination rate constant; NA, not available; OAC, oral allergen challenge; t½, half-life; Tₘₐₓ, time to maximum concentration; V_{d}/F, apparent volume of distribution after non-intravenous administration; Va/F/kg, apparent volume of distribution after non-intravenous administration normalized by body weight.

As shown in Table 9, a greater proportion of participants reached uncorrected epinephrine levels ≥100 pg/mL within the first 10 and 20 minutes following AQST-109 administration compared with IM injection in both single and repeat dose conditions.

TABLE 9
Cumulative proportion of participants to reach 100 pg/mL
of uncorrected epinephrine (Part 1 and 2 combined)

| | Single dose | | | Repeat dose | | |
|---|---|---|---|---|---|---|
| | | | Manual IM | | | Manual IM |
| | | AQST-109 | injection | | AQST-109 | injection |
| Time | AQST-109 | without | without | AQST-109 | without | without |
| point, | with OAC | OAC | OAC | with OAC | OAC | OAC |
| minutes | (n = 24) | (n = 17) | (n = 24) | (n = 24) | (n = 16) | (n = 23) |
| 5 | 7 (29.2) | 0 | 6 (25.0) | 5 (20.8) | 2 (12.5) | 7 (30.4) |
| 10 | 21 (87.5) | 13 (76.5) | 9 (37.5) | 18 (75.0) | 8 (50.0) | 11 (47.8) |
| 15 | 22 (91.7) | 13 (76.5) | 11 (45.8) | 19 (79.2) | 10 (62.5) | 12 (52.2) |
| 20 | 24 (100) | 13 (76.5) | 15 (62.5) | 19 (79.2) | 12 (75.0) | 15 (65.2) |
| 30 | 24 (100) | 14 (82.4) | 18 (75.0) | 23 (95.8) | 13 (81.3) | 19 (82.6) |
| 45 | 24 (100) | 14 (82.4) | 21 (87.5) | 23 (95.8) | 14 (87.5) | 21 (91.3) |
| 60 | 24 (100) | 16 (94.1) | 22 (91.7) | 23 (95.8) | 14 (87.5) | 22 (95.7) |

Data are n (%)

Referring to FIG. 9A and FIG. 9B, oral physiological changes induced by OAC did not compromise the PK performance of AQST-109. For single doses of AQST-109, geometric mean Cmax values were comparable between conditions with OAC (404 pg/mL) and without OAC (320 pg/mL), and median Tmax values were similar (12 minutes vs. 13 minutes).

In the single-dose condition with OAC, reaching 100 pg/mL of uncorrected epinephrine was achieved by 87.5% (n=21/24) of participants by 10 minutes and 100% (n=24/24) of participants by 30-60 minutes, compared with 76.5% (n=13/17) of participants by 10 minutes, 82.5% (n=14/17) by 30 minutes, and 94.1% (n=16/17) by 60 minutes without OAC. In the repeat-dose condition with OAC, 75.0% (n=18/24) of participants reached 100 pg/mL of uncorrected epinephrine by 10 minutes and 94.4% (n=23/24) by 30-60 minutes, compared with 50% (n=8/16) at 10 minutes, 81.3% (n=13/16) at 30 minutes, and 87.5% (n=14/16) at 60 minutes without OAC.

In the single-dose condition, AUC₀₋ₜ and AUC_{0-∞} were lower with OAC versus without OAC. In the repeat-dose condition, AUC₀₋ₜ and AUC_{0-∞} were higher with OAC versus without OAC, suggesting dose- and context-dependent effects on systemic exposure. Repeat dose administration of AQST-109 with OAC yielded the highest Cmax observed (1190 pg/mL, CV 145%); repeat administration without OAC resulted in a Cmax of 505 pg/mL (CV 141%; FIG. 9A and FIG. 9B). Tmax was 12 and 13 minutes with single doses of AQST-109 with and without OAC, respectively and 25 and 28 minutes after repeat-doses of AQST-109 with and without OAC, respectively.

TABLE 10
PK parameters of baseline-corrected epinephrine after single
and repeat doses of AQST-109 with and without and an OAC

| | Single dose | | | | Repeat dose | | | |
|---|---|---|---|---|---|---|---|---|
| | With OAC | | Without OAC | | With OAC | | Without OAC | |
| Parameter | (n = 24) | | (n = 24) | | (n = 24) | | (n = 23) | |
| Cₘₐₓ, pg/mL | 404 | (180) | 320 | (209) | 1190 | (145) | 505 | (141) |
| AUC_{0-5 min}, | 0.5 | (3760) | 0.9 | (104) | 0.3 | (19000) | 4.8 | (2050) |
| h*pg/mL | | | | | | | | |
| AUC_{0-10 min}, | 14.5 | (175) | 9.6 | (225) | 7.0 | (710) | 32.0 | (172) |
| h*pg/mL | | | | | | | | |
| AUC_{0-20 min} | 62.7 | (163) | 43.6 | (258) | 58.4 | (158) | 90.2 | (182) |
| h*pg/mL | | | | | | | | |
| AUC_{0-30 min} | 98.6 | (136) | 72.4 | (194) | 210 | (128) | 155 | (149) |
| h*pg/mL | | | | | | | | |
| AUC_{0-45 min} | 127 | (117) | 109 | (154) | 352 | (116) | 211 | (123) |
| h*pg/mL | | | | | | | | |
| AUC_{0-60 min} | 160 | (101) | 139 | (136) | 436 | (107) | 521 | (75.5) |
| h*pg/mL | | | | | | | | |
| AUC₀₋ₜ h*pg/mL | 306 | (76.8) | 316 | (95.2) | 848 | (83.3) | 550 | (170) |
| AUC_{0-∞} | 425 | (87.2) | 703 | (133) | 1510 | (80.1) | 505 | (141) |
| h*pg/mL | | | | | | | | |
| Median Tₘₐₓ | 12 | | 13 | | 25 | | 28 | |
| (min) | | | | | | | | |
| t_{1/2} (h) | 1.5 | (62.6) | 1.5 | (8.5) | 2.0 | (24.9) | 1.3 | (29.8) |
| Kₑₗ (1/h) | 0.5 | (62.6) | 0.5 | (8.5) | 0.4 | (24.9) | 0.5 | (29.8) |
| CL/F (L/h) | 28200 | (87.2) | 17100 | (133) | 15900 | (80.1) | 43600 | (170) |
| CL/F/kg | 359 | (79.1) | 248 | (164) | 189 | (63.7) | 477 | (153) |
| (L/h/kg) | | | | | | | | |
| V_{d}/F (L) | 55200 | (65.6) | 35700 | (152) | 41000 | (62.7) | 72900 | (189) |
| V_{d}/F/kg (L/kg) | 702 | (65.9) | 519 | (187) | 488 | (53.1) | 797 | (168) |

Data are geometric mean (CV %) unless otherwise noted

### Secondary Endpoint: PD

AQST-109 after OAC Versus Manual IM Injection without OAC

Referring to FIGS. 10A, 10B, 11A, 11B, 12A and 12B, in participants receiving AQST-109 after OAC, single-dose administration produced higher mean (SD) peak changes (Emax) from baseline in SBP, DBP, and HR of 15.5 mmHg (20.7), 11.1 mmHg (15.3) and 15.1 beats/min (12.1), respectively, compared with 7.5 mmHg (12.7), —3.5 mmHg (10.9), and 7.0 beats/min (13.0), respectively, following manual IM injection.

Repeat dosing after OAC resulted in a mean (SD) Emax of 28.1 mmHg (16.5) for SBP, 14.5 mmHg (13.8) for DBP, and 17.6 beats/min (10.7) for HR, which were higher than that observed with IM injection (6.15 mmHg [17.3], —4.4 mmHg [11.9] and 8.2 beats/min [10.8], respectively). Across both single- and repeat-dose conditions, AQST-109 was associated with faster onset (earlier TEmax) and more robust favorable cardiovascular responses compared to manual IM injection.

AQST-109 after OAC Versus AQST-109 without OAC

In the single dose condition with OAC, mean (SD) Emax for SBP, DBP, and HR was 28.3 mmHg (21.6), 10.9 mmHg (15.5), and 16.1 mmHg (13.4); mean (SD) TEmax was 12.0 minutes (10.5), 8.1 minutes (10.8), and 14.1 minutes (10.6). Whereas in the single-dose condition without OAC, mean (SD) Emax for SBP, DBP, and HR was 11.6 mmHg (18.9), 8.2 mmHg (16.4), and 11.5 mmHg (12.8); mean (SD) TEmax was 18.6 minutes (17.1), 14.2 minutes (15.5), and 26.7 minutes (21.6).

In the repeat dose with OAC, mean (SD) Emax for SBP, DBP, and HR was 28.3 mmHg (18.7), 12.3 mmHg (15.1), and 18.6 mmHg (11.3); mean (SD) TEmax was 24.5 minutes (13.7), 19.7 minutes (19.3), and 23.4 minutes (13.1). In the repeat-dose condition without OAC, mean (SD) Emax for SBP, DBP, and HR was 21.7 mmHg (23.6), 10.4 mmHg (16.0), and 9.5 mmHg (14.1); mean (SD) TEₘₐₓ was 25.1 minutes (13.0), 26.7 minutes (18.0), and 24.7 minutes (19.4).

The majority of participants developed moderate symptoms during OAC at screening (n=26/36, 72.2%), the single-dose treatment period (n=18/24, 75.0%), and the repeat-dose treatment period (n=19/24; 79.2%; Table 11). The most common symptoms included pruritus and tingling of the lips, tongue, cheeks, palate, throat, eyes and skin.

TABLE 11
Allergic reaction severity, allergens,
and common symptoms post-OAC

| | Single dose | Repeat dose |
|---|---|---|
| | of AQST-109 | of AQST-109 |
| | (n = 24) | (n = 24) |
| VSR severity | | |

| | | | |
|---|---|---|---|
| Mild discomfort | 0 | 3 | (12.5) |

| | | | | |
|---|---|---|---|---|
| Moderate discomfort | 18 | (75.0) | 19 | (79.2) |
| Severe discomfort | 6 | (25.0) | 2 | (8.3) |
| Allergens | | | | |
| Pineapple | 8 | (33.3) | 6 | (25.0) |
| Apple | 4 | (16.7) | 3 | (12.5) |
| Kiwi | 3 | (12.5) | 4 | (16.7) |
| Cherry | 1 | (4.2) | 2 | (8.3) |
| Banana | 1 | (4.2) | 1 | (4.2) |
| Avocado | 1 | (4.2) | 1 | (4.2) |
| Dragon fruit | 1 | (4.2) | 1 | (4.2) |
| Fig | 1 | (4.2) | 1 | (4.2) |
| Grapefruit | 1 | (4.2) | 1 | (4.2) |
| Lychee | 1 | (4.2) | 1 | (4.2) |
| Tangerine | 1 | (4.2) | 1 | (4.2) |

| | | | |
|---|---|---|---|
| Peach | 1 | (4.2) | 0 |

| | | | |
|---|---|---|---|
| Lemon | 0 | 1 | (4.2) |
| Strawberry | 0 | 1 | (4.2) |

| Post-allergen | | | | |
|---|---|---|---|---|
| challenge symptoms | | | | |
| Cheeks | | | | |
| Itchy | 8 | (33.3) | 1 | (4.2) |
| Tingling sensation | 6 | (25.0) | 2 | (8.30) |
| Swelling | 1 | (4.2) | 1 | (4.2) |
| Dryness | 1 | (4.2) | 0 | |
| Lips | | | | |
| Itchy | 15 | (62.5) | 16 | (66.7) |
| Tingling sensation | 15 | (62.5) | 13 | (54.2) |
| Swelling | 6 | (25.0) | 8 | (33.3) |

| | | | |
|---|---|---|---|
| Dryness | 1 | (4.2) | 0 |

| | | | |
|---|---|---|---|
| Burning | 0 | 1 | (4.2) |

| Numbness | 1 | (4.2) | 0 | |
|---|---|---|---|---|
| Pain | 1 | (4.2) | 1 | (4.2) |
| Ears | | | | |

| | | | |
|---|---|---|---|
| Itchy | 0 | 1 | (4.2) |

| Mouth | | | | |
|---|---|---|---|---|
| Excess saliva | 1 | (4.2) | 0 | |
| Throat | | | | |
| Itchy | 15 | (62.5) | 11 | (45.8) |
| Tingling sensation | 8 | (33.3) | 11 | (45.8) |
| Swelling | 2 | (8.3) | 2 | (8.3) |
| Pain | 0 | | 1 | (4.2) |
| Dryness | 1 | (4.2) | 0 | |
| Tongue | | | | |
| Itchy | 20 | (83.3) | 18 | (75.0) |
| Tingling sensation | 14 | (58.3) | 14 | (58.3) |
| Swelling | 1 | (4.2) | 2 | (8.3) |
| Pain | 2 | (8.3) | 1 | (4.2) |

| | | | |
|---|---|---|---|
| Burning | 0 | 1 | (4.2) |

| Numbness | 1 | (4.2) | 0 | |
|---|---|---|---|---|
| Under tongue | | | | |
| Feeling itchy | 11 | (45.8) | 6 | (25.0) |
| Tingling sensation | 7 | (29.2) | 3 | (12.5) |

| | | | |
|---|---|---|---|
| Swelling | 0 | 1 | (4.2) |

| Roof of mouth | | | | |
|---|---|---|---|---|
| Feeling itchy | 3 | (12.5) | 0 | |
| Tingling sensation | 1 | (4.2) | 1 | (4.2) |
| Bumps | 1 | (4.2) | 0 | |
| Facial/eyes | | | | |
| Itchy eyes | 2 | (8.3) | 4 | (16.7) |
| Lip swelling | 2 | (8.3) | 2 | (8.3) |
| Watery eyes | 1 | (4.2) | 2 | (8.3) |

| | | | |
|---|---|---|---|
| Facial swelling | 0 | 1 | (4.2) |

Gastrointestinal

| | | | |
|---|---|---|---|
| Nausea | 0 | 3 | (12.5) |
| Abdominal discomfort | 0 | 2 | (8.3) |
| Stomach cramps | 0 | 1 | (4.2) |

| | | | | |
|---|---|---|---|---|
| Respiratory | | | | |
| Tickling on back of throat | 5 | (20.8) | 1 | (4.2) |
| Chest tightness | 1 | (4.2) | 1 | (4.2) |

| | | | |
|---|---|---|---|
| Cough | 0 | 1 | (4.2) |

| | | | |
|---|---|---|---|
| Shortness of breath | 1 | (4.2) | 0 |

| | | | |
|---|---|---|---|
| Throat closing | 0 | 1 | (4.2) |

Nasal

| | | | |
|---|---|---|---|
| Nasal congestion | 0 | 1 | (4.2) |
| Running nose | 0 | 1 | (4.2) |
| Sneezing | 0 | 1 | (4.2) |

| Skin | | | | |
|---|---|---|---|---|
| Itching sensation | 4 | (16.7) | 3 | (12.5) |
| Redness | 2 | (8.3) | 2 | (8.3) |
| Neurological | | | | |
| Feeling faint or | 1 | (4.2) | 0 | |
| lightheaded | | | | |
| Cardiovascular | | | | |
| Palpitations/feeling heart | 1 | (4.2) | 0 | |
| racing | | | | |
| Overall | | | | |
| Fatigue | 1 | (4.2) | 0 | |
| Malaise | 1 | (4.2) | 1 | (4.2) |

Data are n (%)

Based on VRS data, the median (range) time from AQST-109 dosing to full symptom resolution at the participant level was 15.0 minutes (2.0-180.0) for single-dose and 10.0 minutes (2.0-180.0) for repeat-dose administration. At the symptom level, resolution occurred even faster: 5.2 minutes (2.0-180.0) and 8.0 minutes (2.0-180.0), respectively (Table 12). In contrast to treatment periods, the median (range) time from maximum symptoms to full symptom resolution at screening was 74.0 minutes (14.5-319.0), highlighting the comparative impact of AQST-109 on OAS symptom resolution.

TABLE 12
Symptom resolution summary in the safety
population (Part 1 and Part 2 combined)

| | Single dose | | Repeat dose | |
|---|---|---|---|---|
| | of AQST-109 | | of AQST-109 | |
| Symptom resolution per VRS | with OAC | | with OAC | |
| data (minutes) | (n = 24) | | (n = 24) | |
| Participant Level - Full | | | | |
| symptom resolution | | | | |
| Time from maximum | 23.5 | (9.3-186.7) | 16.8 | (8.2-187.0) |
| symptoms to symptom | | | | |
| resolution | | | | |
| Time from dosing to symptom | 15.0 | (2.00-180.0) | 10.0 | (2.0-180.0) |
| resolution | | | | |
| Symptom Level - Full | | | | |
| symptom resolution^{a} | | | | |
| Time from maximum | 14.0 | (7.7-186.7) | 15.1 | (8.1-187.0) |
| symptoms to symptom | | | | |
| resolution | | | | |
| Time from dosing to symptom | 5.2 | (2.0-180.0) | 8.0 | (2.0-180.0) |
| resolution | | | | |

Data are median (range)
^{a}Data are based on 164 symptoms in the single-dose cohort and 140 symptoms in the repeat-dose cohorts.
OAC, oral allergen challenge;
VRS, verbal rating scale

### Secondary Endpoint: Safety

In Part 1, all 36 participants experienced at least one TEAE, totaling 176 events, of which 98% were mild (172/176). Of these, 173 were attributed to AQST-109 and 2 to IM injection. In Part 2, all 12 participants experienced a TEAE with a combined 30 TEAEs, which were all considered to be related to AQST-109.

Four TEAEs (administration site paresthesia) were moderate in severity and resolved in ≤4.25 hours. The remaining 172 events were mild, self-limiting and required no medical intervention.

Referring to Table 13, the most commonly reported TEAEs following AQST-109 were administration site AEs (e.g., paresthesia, hypoesthesia, erythema, discomfort, pain, and exfoliation). There were no severe or serious TEAEs, and no deaths. One participant who was receiving the repeat-dose discontinued due to mild, non-cardiac chest pain that began 5 minutes post-dose and resolved within 36 minutes—the second dose was not administered. This event was considered related to AQST-10

TABLE 13
Overall summary of AEs in Part 1 and Part 2 during treatment periods
where AQST-109 was administered in the safety population

| | | | | | Without OAC | |
|---|---|---|---|---|---|---|
| | With OAC | | | | Single | Repeat |
| | Single dose of | | Repeat dose of | | dose of | dose of |
| | AQST-109 | | AQST-109 | | AQST-109 | AQST-109 |
| | Part 1 | Part 2 | Part 1 | Part 2 | Part 1 | Part 1 |
| n (%) | (n = 18) | (n = 6) | (n = 18) | (n = 6) | (n = 17) | (n = 16) |
| TEAE^{b} | 18 (100) | 14 (82.4) | 18 (100) | 15 (93.8) | 1 (5.6) | 1 (5.9) |
| General disorders | 10 (100) | 6 (100) | 17 (94.4) | 6 (100) | 14 (82.4) | 14 (87.5) |
| and administration | | | | | | |
| site conditions | | | | | | |
| Administration site | 15 (83.3) | 4 (66.7) | 16 (88.9) | 5 (83.3) | 10 (58.8) | 14 (87.5) |
| paresthesia | | | | | | |
| Administration site | 11 (61.1) | 3 (50.0) | 10 (55.6) | 6 (100) | 10 (58.8) | 6 (37.5) |
| hypoesthesia | | | | | | |
| Administration site | 8 (44.4) | 1 (16.7) | 8 (44.4) | 1 (16.7) | 2 (11.8) | 1 (6.3) |
| exfoliation | | | | | | |
| Administration site | 7 (38.9) | 0 | 6 (33.3) | 1 (16.7) | 4 (23.5) | 2 (12.5) |
| erythema | | | | | | |
| Administration site | 1 (5.6) | 0 | 6 (33.3) | 0 | 2 (11.8) | 0 |
| discomfort | | | | | | |
| Administration site | 0 | 0 | 2 (11.1) | 0 | 0 | 1 (6.3) |
| pain | | | | | | |
| Administration site | 1 (5.6) | 0 | 0 | 1 (16.7) | 0 | 0 |
| dysesthesia | | | | | | |
| Application site | 0 | 0 | 1 (5.6) | 0 | 0 | 0 |
| ulcer | | | | | | |
| Application site | 0 | 0 | 1 (5.6) | 0 | 0 | 0 |
| pruritus | | | | | | |
| Application site | 1 (5.6) | 0 | 0 | 0 | 0 | 0 |
| swelling | | | | | | |
| Malaise | 0 | 0 | 1 (5.6) | 0 | 0 | 0 |
| Non-Cardiac chest | 0 | 0 | 1 (5.6) | 0 | 0 | 0 |
| pain | | | | | | |
| GI disorders | 2 (11.1) | 2 (33.3) | 7 (38.9) | 1 (16.7) | 0 | 3 (18.8) |
| Abdominal | 0 | 2 (33.3) | 4 (22.2) | 0 | 0 | 1 (6.3) |
| discomfort | | | | | | |
| Dyspepsia | 1 (5.6) | 0 | 2 (11.1) | 0 | 0 | 2 (12.5) |
| Nausea | 1 (5.6) | 0 | 1 (5.6) | 0 | 0 | 1 (6.3) |
| Vomiting | 0 | 0 | 1 (5.6) | 0 | 0 | 1 (6.3) |
| Cardiac disorders | 1 (5.6) | 1 (16.7) | 4 (22.2) | 0 | 0 | 0 |
| Palpitations | 1 (5.6) | 1 (16.7) | 0 | 0 | 0 | 0 |
| Nervous system | 0 | 1 (16.7) | 3 (16.7) | 0 | 0 | 0 |
| disorders | | | | | | |
| Headache | 0 | 1 (16.7) | 1 (5.6) | 0 | 0 | 0 |
| Presyncope | 0 | 0 | 1 (5.6) | 0 | 0 | 0 |
| Tremor | 0 | 0 | 1 (5.6) | 0 | 0 | 0 |

Oral physiological changes induced by allergen exposure did not compromise epinephrine absorption via AQST-109. The single-dose PK curves were similar between the conditions with and without OAC. In both single- and repeat-dose conditions, the primary and secondary PK endpoints were met. Geometric mean values for Cₘₐₓ, AUC_{0-10 min}, AUC_{0-20 min}, AUC_{0-30 min}, AUC_{0-45 min}, and AUC_{0-60 min} were higher following AQST-109 than with manual IM injection, reinforcing the reliability of sublingual absorption even in the presence of oral physiologic changes.

By 10 minutes post dose delivery, a greater proportion of participants treated with AQST-109 with and without OAC had reached the epinephrine physiologic change threshold of 100 pg/mL than with manual IM injection. The median Tmax for AQST-109 during OAC was 12 minutes after a single dose, which is faster than manual IM injection and consistent with previous AQST-109 data.28 These early exposures translated into clinically favorable hemodynamic effects. After a single AQST-109 dose with OAC, mean (SD) peak increases in SBP, DBP, and HR were 15.5 mmHg (20.7), 11.1 mmHg (15.3), and 15.1 bpm (12.1), respectively, exceeding those observed with manual IM injection (7.5 mmHg, —3.5 mmHg, and 7.0 bpm, respectively). With repeat dosing, the effects were even more pronounced, showing sustained pharmacodynamic engagement without exceeding normal physiological ranges.

Importantly, AQST-109 also demonstrated rapid symptom resolution. Using VRS data, the median time to complete symptom resolution was 15.0 minutes (range, 2.0-180.0) following a single dose and 10.0 minutes (range, 2.0-180.0) after repeat dosing. At the individual symptom level, resolution occurred as early as 5.2 minutes and 8.0 minutes, respectively, which was earlier than Tmax in all conditions. In contrast, during screening (baseline OAC without epinephrine), median time from maximum symptoms to full symptom resolution was 74.0 minutes, highlighting the potential therapeutic impact of AQST-109.

The majority of TEAEs were mild and localized to the application site (e.g., paresthesia, hypoesthesia, erythema). Four events (administration site paresthesia) were moderate and self-limiting. Although paresthesia and hypoaethesia are captured as AEs, they are intended effects of the absorption enhancer clove oil which is included in the AQST-109 product to improve absorption of the epinephrine prodrug.31 No severe or serious TEAEs were observed, and the safety profile was maintained even under OAC and repeat-dosing conditions.

This first-of-its-kind study shows that oral mucosal changes during allergic reactions do not impair the PK performance of AQST-109, and its PD responses remained robust. In fact, under OAC, AQST-109 demonstrated rapid systemic absorption, effective hemodynamic response, and consistent resolution of oral allergic symptoms. Its needle-free, sublingual film formulation offers a clinically meaningful alternative to existing IM and intranasal delivery options. Additionally, AQST-109 is portable, easy to use, and suitable for administration under high-stress conditions.

All references cited herein are hereby incorporated by reference herein in their entirety.

Other embodiments are within the scope of the following claims.

What is claimed is:

1. A method of treating an allergy symptom in a subject comprising:
applying a pharmaceutical film to a subject, the pharmaceutical film comprising a polymeric matrix to an oral cavity,
a pharmaceutically active component including epinephrine or a pharmaceutically acceptable salt or ester thereof contained in the polymeric matrix and
an adrenergic receptor interacter contained in the polymeric matrix; and
substantially resolving the allergy symptom earlier than reaching a Cmax of the pharmaceutically active component.

2. The method according to claim 1, wherein pharmaceutical film is applied proximate to a site of the allergy symptom.

3. The method according to claim 1, wherein allergy symptom is substantially resolved prior to Tmax.

4. The method according to claim 1, wherein onset of resolution of the allergy symptom occurs in 20 minutes or less.

5. The method according to claim 1, wherein onset of resolution of the allergy symptom occurs in 15 minutes or less.

6. The method according to claim 1, wherein onset of resolution of the allergy symptom occurs in 12 minutes or less.

7. The method according to claim 1, wherein onset of resolution of the allergy symptom occurs in 10 minutes or less.

8. The method according to claim 1, wherein onset of resolution of the allergy symptom occurs in 8 minutes or less.

9. The method according to claim 1, wherein onset of resolution of the allergy symptom occurs in 5 minutes or less.

10. The method according to claim 1, wherein onset of resolution of the allergy symptom occurs in 2 minutes or less.

11. The method according to claim 1, further comprising applying a repeat dose of the pharmaceutical film.

12. The method according to claim 1, wherein the allergy symptom is an oral allergy symptom.

13. The method according to claim 1, wherein the allergy symptom includes an oral allergy symptom, body allergy symptom, or systemic allergy symptom.

14. The method according to claim 11, further comprising maintaining a consistent pharmacokinetic profile with a repeat dose.

15. The method according to claim 1, wherein the treatment elicits a desired pharmacodynamic response in systolic blood pressure, diastolic blood pressure and pulse with and without an allergen.

16. The method according to claim 1, wherein the adrenergic receptor interacter is a permeation enhancer that includes a phenylpropanoid, farnesol, a capric/caprylic triglyceride polyethylene glycol derivative or linoleic acid.

17. The method according to claim 1, wherein the adrenergic receptor interacter is eugenol or eugenol acetate, a cinnamic acid, cinnamic acid ester, cinnamic aldehyde, hydrocinnamic acid, chavicol, or safrole.

18. The method according to claim 1, wherein the adrenergic receptor interacter is a phytoextract.

19. The method according to claim 18, wherein the phytoextract further includes an essential oil extract of a clove plant, an essential oil extract of a leaf of a clove plant, an essential oil extract of a flower bud of a clove plant, or an essential oil extract of a stem of a clove plant.

20. The method according to claim 18, wherein the phytoextract is a synthetic or biosynthetic compound.

21. The method according to claim 1, wherein a Cmax is between 250-500 pg/mL.

22. The method according to claim 1, wherein the Tmax is 15 minutes.

23. The method according to claim 1, wherein the Tmax is 12 minutes.

24. The method according to claim 1, wherein the Tmax is 10 minutes.

25. The method according to claim 1, wherein the Tmax is 8 minutes.

26. The method according to claim 1, wherein the Tmax is 5 minutes.
